(19) United States
(12) Patent Application Publication
(10) Pub. No.: US 2007/0066519 A1
(43) Pub. Date: Mar. 22, 2007

(54) Use of human growth hormone in multiple system atrophy

(75) Inventor: Bengt-Ake Bengtsson, Askim (SE)

Correspondence Address: HOWREY LLP, C/O IP DOCKETING DEPARTMENT, 2941 FAIRVIEW PARK DR, SUITE 200, FALLS CHURCH, VA, 22042-2924, US

(73) Assignee: ARES TRADING S.A., Aubonne (CH)

(21) Appl. No.: 10/595,076

(22) PCT Filed: Jul. 29, 2003
(86) PCT No.: PCT/EP03/50348

§ 371 Date: Sep. 7, 2006

(51) Int. Cl. A61K 38/30; A61K 38/17; A61K 48/00

(52) U.S. Cl. 514/12; 514/44

(57) ABSTRACT

The invention relates to the use of a substance, which binds to and initiates signaling of the human growth hormone (hGH) receptor or a substance, which stimulates release or potentiates the activity of endogenous hGH, for treatment and/or prevention of Parkinsonism-Plus Syndromes. In particular, the invention relates to the use of hGH for the treatment and/or prevention of Multiple System Atrophy.

### FIELD OF INVENTION

The present invention is in the field of neurologic diseases. More specifically, it relates to the use of human growth hormone for the manufacture of a medicament for treatment and/or prevention of Parkinsonism-Plus Syndromes, and in particular Multiple System Atrophy (MSA).

### BACKGROUND OF THE INVENTION

Human growth hormone (hGH), also known as somatropin (INN) or somatotropin, is a protein hormone produced and secreted by the somatotropic cells of the anterior pituitary. Human growth hormone plays a key role in somatic growth in childhood and in metabolism in adulthood through its effects on the metabolism of proteins, carbohydrates and lipids.

Human growth hormone is a single polypeptide chain of 191 amino acids (Bewly et al, 1972) having two disulfide bonds, one between Cys-53 and Cys-165, forming a large loop in the molecule, and the other between Cys-182 and Cys-189, forming a small loop near the C-terminus. The DNA sequence that confirmed the amino acid sequence was reported by Martial et al (1979). Purified hGH is a white amorphous powder in its lyophilized form. It is readily soluble (concentrations>10 mg/L) in aqueous buffers at pH in a range of 6.5 to 8.5.

In solution, hGH exists predominantly as a monomer, with a small fraction as dimers and higher molecular weight oligomers. Under certain conditions, hGH can be induced to form larger amounts of dimers, trimers and higher oligomers.

Several derivatives of hGH are known, including naturally-occurring derivatives, variants and metabolic products, degradation products primarily of biosynthetic hGH and engineered derivatives of hGH produced by genetic methods. One example of a naturally-occurring derivative of hGH is GH-V, a variant of growth hormone found in the placenta. Other members of the gene locus are described in Chen et al (1989).

Methionyl hGH was the first form of hGH to be produced through recombinant DNA technology. This compound is actually a derivative of hGH having one additional methionine residue at its N-terminus (Goeddel et al, 1979).

A naturally-occurring variant of hGH called 20-K-hGH has been reported to occur in the pituitary as well as in the bloodstream (Lewis et al, 1978; Lewis et al, 1980). This compound, which lacks the 15 amino acid residues from Glu-32 to Gln-46, arises from an alternative splicing of the messenger ribonucleic acid (DeNoto et al, 1981). This compound shares many, but not all of the biological properties of hGH.

20-K-hGH is made in the pituitary and secreted into the blood. It makes up about 5% of growth hormone output of adults, and about 20% of growth hormone output of children. It has the same growth promoting activity as 22 kD growth hormone, and has been reported to have equal to or greater the amount of lipolytic activity as the 22 kD form. It binds to growth hormone receptors with equal affinity as the 22 kD growth hormone, and has one tenth the lactogenic (prolactin-like) bioactivity as the 22 kD hormone. Unlike 22 kD, the 20-k-hGH has weak anti-insulin activity.

A number of derivatives of hGH arise from proteolytic modifications of the molecule. The primary pathway for the metabolism of hGH involves proteolysis. The region of hGH around residues 130-150 is extremely susceptible to proteolysis, and several derivatives of hGH having nicks or deletions in this region have been described (Thorlacius-Ussing, 1987). This region is in the large loop of hGH, and cleavage of a peptide bond there results in the generation of two chains that are connected through the disulfide bond at Cys-53 and Cys-165. Many of these two-chain forms are reported to have increased biological activity (Singh et al, 1974). Many derivatives of human growth hormone have been generated artificially through the use of enzymes. The enzymes trypsin and subtilisin, as well as others, have been used to modify hGH at various points throughout the molecule (Lewis et al, 1977; Graff et al, 1982). One such derivative, called two-chain anabolic protein (2-CAP), was formed through the controlled proteolysis of hGH using trypsin (Becker et al, 1989). 2-CAP was found to have biological properties very distinct from those of the intact hGH molecule, in that the growth-promoting activity of hGH was largely retained and most of the effects on carbohydrate metabolism were abolished.

Asparagine and glutamine residues in proteins are susceptible to deamidation reactions under appropriate conditions. Pituitary hGH has been shown to undergo this type of reaction, resulting in conversion of Asn -152 to aspartic acid and also, to a lesser extent, conversion of Gln-137 to glutamic acid (Lewis et al, 1981). Deamidated hGH has been shown to have an altered susceptibility to proteolysis with the enzyme subtilisin, suggesting that deamidation may have physiological significance in directing proteolytic cleavage of hGH. Biosynthetic hGH is known to degrade under certain storage conditions, resulting in deamidation at a different asparagine (Asn-149). This is the primary site of deamidation, but deamidation at Asn-152 is also seen (Becker et al, 1988). Deamidation at Gln-137 has not been reported in biosynthetic hGH.

Methionine residues in proteins are susceptible to oxidation, primarily to the sulfoxide. Both pituitary-derived and biosynthetic hGH undergo sulfoxidations at Met-14 and Met-125 (Becker et al, 1988). Oxidation at Met-170 has also been reported in pituitary but not biosynthetic hGH. Both desamide hGH and Met-14 sulfoxide hGH have been found to exhibit full biological activity (Becker et al, 1988).

Truncated forms of hGH have been produced, either through the actions of enzymes or by genetic methods. 2-CAP, generated by the controlled actions of trypsin, has the first eight residues at the N-terminus of hGH removed. Other truncated versions of hGH have been produced by modifying the gene prior to expression in a suitable host. The first 13 residues have been removed to yield a derivative having distinctive biological properties (Gertler et al, 1986) in which the polypeptide chain is not cleaved.

Although human growth hormone was originally obtained from pituitary glands of cadavers, these preparations were not electrophoretically homogeneous, and antibodies appeared in the serum of patients treated with preparations of the order of 50% purity, the immunogenicity being attributed to inactive components. Recombinant DNA technology permitted production of an unlimited supply of hGH in a number of different systems. Purification of hGH from the culture medium is facilitated by the presence of only low amounts of contaminating proteins. In fact, it has been shown that hGH can be purified on a laboratory scale by a single purification step on a reversed-phase HPLC column (Hsiung et al (1989).

Recombinant human growth hormone, rhGH, is produced by Serono International S.A. as SEROSTIM®, which product has been given accelerated FDA approval for treating weight loss and wasting in AIDS patients. SAIZEN® is recombinant human growth hormone indicated for GH deficiency in children, for Turner syndrome in girls, as well as chronic renal failure in children. PROTROPIN®, produced by Genentech, Inc. (South San Francisco, Calif.), differs slightly in structure from natural sequence hGH, having an additional methionine residue at the N-terminus. Recombinant hGH is generally marketed as vials containing hGH plus additional excipients, e.g., glycine and mannitol, in a lyophilized form. A companion diluent vial is provided, allowing the patient to reconstitute the product to the desired concentration prior to administration of the dose. Recombinant hGH can also be marketed in other well-known manners, such as prefilled syringes, etc.

In general, no significant differences have been observed in the pharmacokinetics or biological activities of recombinant natural sequence hGH, recombinant N-methionyl-hGH, or pituitary-derived material in humans (Moore et al, 1988; Jorgensson et al, 1988).

During development, endogenous growth hormone promotes numerous key events and functions and acts directly or indirectly on virtually every tissue in the body. Therefore redundant growth hormone receptors in the mature central nervous system (CNS) may provide novel therapeutic strategies. Furthermore, GH has metabolic actions that are important in many species long after major statural growth has been accomplished. Although the actions of GH were long thought to be mediated entirely via the generation of hepatic insulin-like growth factor-1 (IGF-1), it is now clear that GH also has direct effects in many tissues, acting in concert with locally generated IGF-1 (and probably many other growth factors) in addition to IGF-1 from the circulation.

Although growth hormone is mainly synthesized in the pituitary there is also a widespread ectopic production in different areas of the brain (Johansson et al., 2000). In addition, there is an abundant occurrence of growth hormone receptors, IGF-1 and IGF-1 receptors. During growth hormone treatment in the CNS, there are also changes in CSF levels of GH, GH-dependent factors and neurotransmitters. This may indicate that neuro-endocrine mechanisms are involved in the improvement of physical as well as psychological well being observed during growth hormone treatment in growth hormone deficient adults.

Early experimental findings showed that GH increases the plasticity of the brain. For instance, studies with radiolabeled GH suggested the presence of specific binding sites in CNS, but these were of low abundance and principally identified in hypothalamic regions and choroid plexus although a much wider distribution has been assumed (Harvey et al., 1993). A physiological effect of GH in the CNS is inhibition of its own release, as part of an autofeedback circuit (Tannenbaum, 1980).

Patients with Growth Hormone Deficiency (GHD) have a higher level of perceived health problems. As a group, these patients are less energetic, less physically mobile and more socially isolated (Johannsson et al., 2000). Furthermore, they sleep less well and have a subnormal memory performance. Complaints in these patients have mainly been of tiredness, low energy and lack of initiative, lack of concentration, memory difficulties and irritability.

During GH treatment, energy and mood have improved, as well a s memory. The changes observed indicate a normalization, since. similar levels of energy, mood and memory are observed in a healthy population.

GH deficiency, and more particularly GH treatment, are associated with a variety of changes in the major central neurotransmitters, their biosynthetic enzymes, or their receptors (Andersson et al., 1983) but a physiological role for endogenous GH acting directly on these systems, has not yet been established. Although largely overlooked, GH has a number of neurotrophic actions (stimulating neuronal and glial proliferation, increasing myelination, and increasing brain size), whereas GH deficiency is associated with deficits in brain development (Elias Eriksson, 1985).

In a one-month double-blind placebo controlled study it has previously been shown that GH treatment in GH deficient adults causes a mean ten-fold increase in GH in the CSF. In addition, the mean increase in CSF IGF-1 concentrations was about 50%, the CSF dopamine metabolite homovanillic acid (HVA) concent ration (Harvey et al., 1993) decreased and the CSF β-endorphin immunoreactivity increased during GH treatment. The fall in the CSF HVA concentration indicates that GH affects the dopamine turnover in the CNS, which is in line with previous animal studies and a study by Burman et al (Burman et al., 1995). It seems likely that these neuro-endocrine changes may be involved in the improvement in psychological well being during GH treatment of GH deficient adults.

Parkinsonism-Plus syndromes, also called Parkinsonian-Plus or simply Parkinson-Plus syndromes, form a group of diseases, which are distinct from classical Parkinson's Disease. Parkinsonism-Plus syndromes include the following diseases: Progressive Supranuclear Palsy (PSP), Multiple System Atrophy (MSA,) Parkinson's-amyotrophic lateral sclerosis-dementia of Guam, Generalized Lewy body disease, Corticobasal ganglionic degeneration (CGD), Alzheimer's/Parkinson's overlap syndrome, Huntington's disease: rigid variant, Hallervorden-Spatz disease Gerstmann-Strausler syndrome.

For progressive PSP, the onset of symptoms usually occur between 55 and 70 years of age, while onset before 50 is rare. Different sets of clinical criteria have been proposed since the first description of the disease. The two most specific symptoms on which the clinical diagnosis are based on are the supranuclear gaze palsy, including the inability to move the gaze towards a tacical stimulus, and the postural instability with early falling. Important exclusion criteria such as a good and sustained effect by levodopa therapy, alien hand syndrome, hallucinosis, cortical dementia, cerebellar symptoms and early dysautonomic symptoms, were further established in the criteria proposed by Litvan et al. in 1996. The characteristic microscopic pathology has been reported as neurofibrillary tangles, neuropil threads and neuronal loss in the globus pallidus, sub stancia nigra, superior colliculus, periaqueductal grey, pretectal areas, brainstem and medulla.

The pathogenesis of PSP may be related to the abnormal metabolism of cytoskeletal components (neurofilaments) with an accumulation of tau protein in neurons and glial cells. PSP is considered to be a sporadic disorder, but a hereditary cause, such as a genetic variation related to the tau gene. Early signs are bilateral bradykinesia, with a rigidity of the axial type that can even be absent in the extremities. The course of the disease is always progressive and appears to be less variable than in Parkinson's Disease and MSA. Men are more affected by the disease and tend to have a worse prognosis whereas females and patients with an earlier onset of disease may have a somewhat better prognosis. After some years of disease, the clinical picture often becomes more evident. Eye movements become slower, first horizontally, then vertically. The patient has difficulty focusing and meeting the eye of the clinician and has a staring gaze due to the involuntary persistence of ocular fixation. Later in the disease, supranuclear ophthalmoparesis with paralysis of the down gaze and square wave jerks is typically found in PSP. The gait becomes awkward with the abduction of the arms, pivoting when turning and a straight body posture. A typical “astonished” expression on the face is described and the patient often denies severe problems due to a frontal lobe dysfuncion. Severe postural instability occurs most prominently in the middle of the course of the disease and may be a dominant problem. Multiple traumas with fractures of the arms and legs, even lethal trauma due to falling may occur.

Corticobasal degeneration (CBD) was first described in 1968 and is regarded by many as the most difficult diagnosis to make in early disease. CBD is a rare condition and reliable prevalence data are not available. The mean age of clinical onset is 60-65 years, The clinical picture with regard to Parkinson's disease often differs gradually with time. The pathology is distinguished from Parkinson's disease with the presence of large swollen achromatic neurons as a main finding. Atrophy of both the cortical and basal structures, i.e. the basal ganglia, substantia nigra and brainstem, is found. The onset of symptoms is usually located in one of the upper limb. The lower limb on the ipsi-lateral side is affected before the contralateral side becomes involved. The manifestations can be divided into three categories: symptoms indicating affection of the 1) cortex, 2) basal ganglia or 3) involvement of other structures. Dystonia is frequently seen. When unilateral action tremor is present, the picture may be confused with that of an essential tremor. With time, tremor becomes myoclonic. Apraxia can sometimes be an early presentation, giving clues to the diagnosis, but a vascular lesion may produce a similar picture. After some years, the characteristic “alien limb” phenomenon is seen. Cortical sensory loss is a late feature, which is useful in distinguishing the syndrome from Parkinson's disease. Other symptoms such as supranuclear gaze palsy, dysarthria, dysphagia, pyramidal symptoms are late manifestations of the disease. The asymmetry of symptoms is, however, persistent and the risk of confusing the syndrome with PSP is therefore small.

Diffuse Lewy body disease (DLBD) has emerged as the second most common cause of degenerative dementia in the elderly after Alzheimer's disease (AD). However, the clinical differentiation between these disorders is difficult. In the case of DLBD, gait impairment, rigidity, resting tremor early in disease have been reported, as well as psychosis and dementia. Complex visual hallucinations at an early stage of the disease are particularly characteristic of DLBD. In addition to progressive cognitive decline, two of the following criteria are required for a diagnosis of probable DLBD and one for a possible diagnosis. 1. Fluctuating cognition with pronounced variations in attention and alertness, 2. Recurrent, typically well-formed, visual hallucinations and 3. motor features of Parkinsonism. Supportive features, not required for the diagnosis, include repeated falls, syncope, transient loss of consciousness, neuroleptic sensitivity, systematized delusions and hallucinations in other modalities.

Multiple System Atrophy (MSA) is a neurodegenerative disorder in which degeneration in brain regions leads to impaired control of movement, balance, blood pressure and sexual and urinary tract function.

MSA is a distinct clinicopathological entity (Gilman et al., 1998). Patients are designated MSA-P if Parkinsonian features predominate or MSA-C if cerebellar features predominate. MSA-M is a mixed sub-type which include patients with pyramidal or cerebellar signs.

MSA typically presents in the fifth to seventh decade of life with a slightly higher incidence in males. Patients usually have autonomic nervous system dysfunction first. Genitourinary dysfunction is the most frequent initial complaint in women while impotence is the most frequent initial complaint in men. Orthostatic hypotension is common and may cause dizziness, dimming of vision, head or neck pain, yawning, temporary confusion, slurred speech and if the hypotension is severe the patient may faint upon arising from a recumbent position.

MSA differs from classical Parkinson's disease in some important aspects: Early onset (5 to 10 years younger than Parkinson patients), marginal response on L-dopa treatment, rapidly progressing and survival is rarely more than 7 years after diagnosis. While in Parkinson's disease the brunt of damage is primarily in one system, the nigrostriatal pathway, in MSA multiple neuronal systems are damaged. The incidence of MSA is 5-15:100 000, and may account for 10% of patients with clinically idiopathic Parkinsons. The cause is unknown and there is no known cure.

It is hypothesised that the symptoms in MSA are related to the progressive degeneration of neurons (Holmberg et al., 1998). MSA patients have been shown to have elevated levels of markers for neurodegeneration in the cerebrospinal fluid.

Multiple System Atrophy (MSA) is perhaps the most frequent differential diagnosis to Parkinson's Disease (Parkinson's Disease) at a movement disorder unit and could comprise up to 10% of all patients presenting with Parkinsonism (Quinn 1989). MSA consists of 3 parts: Shy-Drager syndrome, Striatonigral degeneration, and Olivopontocerebellar atrophy. As mentioned above, Multiple System Atrophy (MSA) is a neurodegenerative disorder in which degeneration in brain regions leads to impaired control of movement, balance, blood pressure and sexual and urinary tract function.

The onset of the disease is variable. The earliest cases present in the fourth decade of life, whereas the mean onset was 50 years of age in a study of autopsy-confirmed cases (Wenning 1996). A slight male preponderance is seen.

The term MSA comprises a chronic degenerative disorder producing different combinations of symptoms from the basal ganglia, pyramidal pathways, cerebellum, brainstem and autonomic nervous system. The nomenclature of the different manifestations of MSA has been variable and has probably delayed the awareness of the disease. For patients with predominant Parkinsonism symptoms, the term MSA-SND has been suggested, whereas MSA-OPCA could be used when cerebellar predominance is found (Quinn 1989). During the 1990s, this nomenclature was further discussed. MSA-P and MSA-C are also terms that have been proposed as description of the various expressions of the disease with Parkinsonism and cerebellar predominance respectively. Different sets of diagnostic criteria have been proposed (Quinn 1989). No systematic evaluation of these criteria has so far been made, although the accuracy of the clinical diagnosis of MSA has been estimated retrospectively among neurologists (Litvan et al., 1997). A specificity of over 90% to identify MSA was already found at the first clinical evaluation, although the sensitivity remained low in spite of repeated evaluations.

The neuropathological findings comprise specific gliocytoplasmatic inclusion bodies, gliosis and nerve cell loss in the putamen, substantia nigra, basis pontis, inferior olives, cerebellar folia, spinal cord intermediolateral column and Onuf's nucleus (Daniel, 1999). Further regions affected by the disease are locus ceruleus, dorsal vagal nucleus, pyramidal tract and anterior horn cells (Wenning, 1996, Wenning, 1997, Van der Ecken et al., 1960).

MSA typically is a sporadic disease (Bandmann et al., 1997). The course of the disease is heterogeneous, which further reflects the various designations of its appearance.

Autonomic dysfunction is a common finding in MSA and may present as presyncopal episodes, incontinence or sexual dysfunction (Wenning, 1994). Other less specific signs of autonomic failure are dizziness, muscle pain, constipation and fatigue. Cold dusky hands should also raise the suspicion of MSA (Klein et al., 1997). The prognosis becomes poor when falls in blood pressure with repeated syncope has developed. More than 40% of patients become wheelchair bound within five years due to the movement disturbance.

The individual prognosis may vary substantially. MSA patients with a disease duration of more than 20 years have been reported (Wenning 1997), whereas the mean survival time in autopsy-confirmed cases, where a bias for more aggressive cases is likely, is reported to be six to nine years from onset (Wenning 1997, Wenning 1994).

Depression and anxiety are commonly seen both early and late in the course of all the Parkinsonism-Plus syndromes, often with a good response to antidepressants.

The diagnostic assistance from laboratory methods has so far only been evaluated in relatively small study populations of clinically diagnosed or post-mortem-proven cases. Methods with a high sensitivity (number of true-positively predicted diagnoses by the test/total number of patients with the disease) and specificity (number of true negatively predicted diagnoses by the test/number of patients without the disease), as well as those that give an objective quantification of data, are promising and can aid the clinical diagnosis.

MRI findings are commonly reported in patients with MSA, where this technique is sometimes useful for distinguishing MSA-P from Parkinson's Disease and Supranuclear Palsy (PSP). In particular, the combination of hypo-and hyperintense putaminal signal changes on T2-weighted MRI sequences has been regarded as highly specific for the diagnosis, although the sensitivity is low (Schrag, 1998; Kraft et al., 1999). These findings could be useful in distinguishing some MSA-P patients with a positive levodopa response from those with Parkinson's Disease. Isolated hypointense changes in the putamen are also common in MSA but are less specific, as they are also reported in Parkinson's Disease and PSP (Schrag et al., 1998). Infratentorial abnormalities have also been reported; transverse pontine fibres, a pontine hyperintensity resembling a cross and a diffuse hyperintensity of the middle cerebellar peduncles are seen with coexisting atrophy (Schrag et al., 1998). These infratentorial changes can only serve as support for the clinical diagnosis as they tend to be preceded by the typical combination of cerebellar and brain-stem-related symptoms, which already distinguish MSA from Parkinson's Disease and PSP.

The clinical response to levodopa is an important feature in every set of diagnostic criteria for Parkinsonism disorders. Several authors have proposed single-dose testing with apomorphine (Hughes, 1990) or levodopa (Hughes et al., 1991; Rossi et al., 2000), as tools for the differential diagnosis and prediction of long-term treatment effects.

A levodopa test with Posturo-Locomotion-Manual recordings can provide further diagnostic information. This test is a complex whole-body motor task that requires that the patient is able to walk unaided.

Markers of neuronal degeneration and gliosis have recently emerged as potential methods for diagnosis, prognosis and the evaluation of treatment (Rosengren et al., 1994; Rosengren et al., 1996). Neuronal degeneration and glial reactivity in various conditions with acute or chronic damage to the Central Nervous System (CNS) can be identified by high concentrations of various brain-specific proteins in the cerebrospinal fluid (CSF) (Rosengren et al., 1999). Moreover, they provide data that is essentially unrelated to the clinical diagnostic criteria for the various Parkinsonism disorders.

The neurofilament is a major structural element of neurons where it maintains axon calibre, neuronal size and shape. High levels of neurofilament have been detected in the CSF of patients with amyotrophic lateral sclerosis and Alzheimer's disease, as well as in other neuro-degenerative disorders, and it has been suggested that NFL in the CSF can be used as a marker of axonal degeneration (Rosengren, 1996).

The GFA protein is a major astroglial protein expressed mainly in the fibrillary astrocytes. The CSF concentration of GFA protein is influenced by different pathological states of the brain. High levels of GFA protein have been observed as a consequence of acute CNS injury and the disintegration of astroglial cells (Rosengren et al., 1994). In chronic brain disorders with gliosis, such as dementia, multiple sclerosis and chronic encephalopathies, GFA protein levels were increased (Rosengren et al., 1994, Rosengren et al., 1995). It has therefore been suggested that GFA protein can be used as a CSF marker of both CNS tissue disintegration and astrogliosis (Rosengren et al., 1994).

Kimber et al. (1997) reported that a clonidine diagnostic test was useful for the identification of MSA patients. Repeated measurements of growth hormone (GH) were made after an intravenous injection of clonidine. A group of MSA patients were found to have a significantly decreased response to clonidine compared with Parkinson's Disease patients, and the test was suggested to be an indicator of central autonomic failure, indicating a loss of medullary catecholaminergic neurons innervating the hypothalamus. In a further study (Kimber et al., 2000), GH concentrations after clonidine injection were found to be significantly lower in a group of MSA patients compared both with controls and with a group of PSP patients, thus providing a diagnostic test for the differentiation of MSA from other neurodegenerative diseases.

### SUMMARY OF THE INVENTION

The present invention is based on the finding that human growth hormone has a beneficial effect on patients suffering from a disease belonging to the a Parkinsonism-Plus Syndromes, namely Multiple System Atrophy. Therefore, the invention relates to the use of a substance, which binds to and initiates signaling of the human growth hormone (hGH) receptor or a substance, which stimulates release or potentiates the activity of endogenous hGH, for treatment and/or prevention of a Parkinsonism-Plus Syndrome. Said substance is particularly suitable for the treatment and/or prevention of Multiple System Atrophy (MSA), which is an as yet untreatable disease clearly delimited from classical Parkinson's Disease.

The invention further relates to the use of a nucleic acid molecule comprising the coding sequence of a substance, which binds to and initiates signaling of the human growth hormone (hGH) receptor or a substance, which stimulates release or potentiates the activity of endogenous hGH, for treatment and/or prevention of a Parkinsonism-Plus Syndrome, in particular MSA.

The use of a vector inducing and/or enhancing endogenous production of a substance, which binds to and initiates signaling of the human growth hormone (hGH) receptor or a substance, which stimulates release or potentiates the activity of endogenous hGH, for treatment and/or prevention of a Parkinsonism-Plus Syndrome, in particular MSA, is also within the present invention.

The invention further relates to a cell that has been genetically modified to produce a substance, which binds to and initiates signaling of the human growth hormone (hGH) receptor or a substance, which stimutates release or potentiates the activity of endogenous hGH, for treatment and/or prevention of a Parkinsonism-Plus Syndrome, in particular MSA.

### DETAILED DESCRIPTION OF THE INVENTION

The present invention is based on the finding that Multiple System Atrophy (MSA), is a disorder belonging to the Parkinsonism-Plus Syndromes, may be treated by administration of an effective amount of human growth hormone.

Therefore, the invention relates to the use of a substance, which binds to and initiates signaling of the human growth hormone (hGH) receptor, or a substance, which stimulates release or potentiates the activity of endogenous hGH, for the preparation of a medicament for treatment and/or prevention of a Parkinsonism-Plus Syndrome.

In a preferred embodiment of the invention, the Parkinsonism-Plus Syndrome is selected from the group consisting of Progressive Supranuclear Palsy (PSP), Multiple system atrophy (MSA), Parkinson's-amyotrophic lateral sclerosis-dementia of Guam, Generalized Lewy body disease, Corticobasal ganglionic degeneration, Alzheimer's/Parkinson's overlap syndrome, Huntington's disease: rigid variant, Hallervorden-Spatz disease, and Gerstmann-Strausler syndrome.

These diseases, along with possibilities to diagnose them, and differentiate them from each other, and from other neurological disorders, have been described in detail in the “Background of the invention” above.

In a particularly preferred embodiment, the Parkinsonism-Plus Syndrome is Mutiple System Atrophy. MSA is a distinct clinicopathological entity (Gilman et al., 1998) that is e.g. characterized by the fact that unlike patients suffering from Parkinson's Disease, MSA patients generally display only a marginal response to levodopa (L-dopa) treatment. Tests for assessing clinical response to levodopa have been described above in the “Background of the invention”. The invention may be any type of MSA, including MSA-P, MSA-C and MSA-M, MSA-SND or MSA-OPCA, as described above in the “Background of the invention”.

The terms “treatment” and “prevention”, as used herein, should be understood as partially or totally preventing, inhibiting, attenuating, ameliorating or reversing one or more symptoms or cause(s) of Parkinson-Plus syndrome, as well as symptoms, diseases or complications accompanying Parkinson-Plus syndrome. When “treating” Parkinson-Plus syndrome, the substances according to the invention are given after onset of the disease, “prevention” relates to administration of the substances before signs of disease is diagnosed or manifest in the patient.

In a preferred embodiment of the invention, said substance is selected from:
a) human growth hormone;
b) a fragment of (a) which has agonistic activity on the hGH receptor;
c) a variant of (a) or (b) which has at least 70% sequence identity with (a) or (b) and which has agonistic activity on the hGH receptor;
d) a variant of (a) or (b) which is encoded by a DNA sequence which hybridizes to the complement of the native DNA sequence encoding (a) or (b) under moderately stringent conditions and which has agonistic activity on the hGH receptor; or
e) a salt or functional derivative of (a), (b), (c) or (d) which has agonistic activity on the hGH receptor.

The term “human growth hormone”, or “hGH”, as used in the present invention, is intended to include the naturally-occurring and synthetic derivatives, as noted above, including, without limitation, both the 20 kD and the 22 kD human growth hormone, GH-V, and other members of the growth hormone gene locus, as described in detail in the “Background of the invention”.

The hGH may be naturally-occurring human growth hormone, or it may preferably be recombinant hGH. Recombinant GH may be expressed in any suitable host, either a prokaryotic, or a eukaryotic host. E. coli is a host particularly suitable for expression of hGH, for instance. Yeast, insect, or mammalian cells are further suitable for expression of recombinant growth hormone. Preferably, the hGH is expressed in human or animal cells, e.g. in Chinese Hamster Ovary (CHO) cells.

The term “hGH” or “growth hormone”, as used herein, also includes functional derivatives, fragments, variants, analogs, or salts which retain the biological activity of growth hormone, i.e., which act as agonists to the growth hormone receptor. In other words, they are capable of binding to the growth hormone receptor to initiate the signaling activity of the receptor.

The term “functional derivatives”, or “chemical derivatives”, as used herein covers derivatives which may be prepared from the functional groups which occur as side chains on the residues of the N- or C-terminal groups, by means known in the art, and are included in the invention as long as they remain pharmaceutically acceptable, and do not destroy the biological activity of hGH as described herein, i.e., the ability to bind the hGH receptor and initiate receptor signaling, and do not confertoxic properties on compositions containing it. Derivatives may have chemical moieties, such as carbohydrate or phosphate residues, provided such a derivative retains the biological I activity of hGH and remains pharmaceutically acceptable.

For example, derivatives may include aliphatic esters of the carboxyl groups, amids of the carboxyl groups by reaction with ammonia or with primary or secondary amines, N-acyl derivatives or free amino groups of the amino acid residues formed with acyl moieties (e.g., alkanoyl or carbocyclic aroyl groups) or O-acyl derivatives of free hydroxyl group (e.g., that of seryl or threonyl residues) formed with acyl moieties. Such derivatives may also include for example, polyethylene glycol side-chains, which may mask antigenic sites and extend the residence of the molecule in body fluids.

Of particular importance is a growth hormone that has been derivatized or combined with a complexing agent to be long lasting. Therefore, a preferred embodiment of the invention relates to PEGylated versions of human growth hormone. Growth hormones genetically engineered to exhibit long lasting activity in the body, are also examples for hGH derivatives within the scope of the present invention. hGH that is acetylated at the N-terminus has been isolated and identified (Lewis et al, 1979). It is not clear if acylation serves a regulatory role or is simply an artifact of the purification. However, it is expected that this molecule exhibits anti-MSA activity in a similar fashion to other hGH derivatives. Therefore, in a preferred embodiment, the invention relates to human growth hormone which is acetlyated at its N-terminus.

Further preferred chemical derivatives of the present invention comprise deaminated hGH, or hGH which is sulfoxidized at one or more methionine residues.

Preferably, the medicament according to the invention comprises a dimer of human growth hormone selected from the group consisting of a disulfide dimer connected through interchain disulfide bonds, a covalent irreversible non-disulfide dimer, a non-covalent dimer, and mixtures thereof.

The term “salts” herein refers to both salts of carboxyl groups and to acid addition salts of amino groups of the hGH molecule or analogs thereof. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids, such as, for example, hydrochloric acid or sulfuric acid, and salts with organic acids, such as, for example, acetic acid or oxalic acid. Of course, any such salts must retain the biological activity of hGH relevant to the present invention, i.e., the ability to bind to the hGH receptor and initiate receptor signaling.

In a further preferred embodiment, the invention relates to fragment of human growth hormone.

A “fragment” of the growth hormone according to the present invention refers to any subset of the molecule, that is, a shorter peptide, which retains the desired biological activity. Fragments may readily be prepared by removing amino acids from either end of the hGH molecule and testing the resultant for its properties as an hGH receptor agonist. Proteases for removing one amino acid at a time from either the N-terminal or the C-terminal of a polypeptide are known, and so determining fragments which retain the desired biological activity involves only routine experimentation.

Preferably, hGH fragments in accordance with the present invention may have internal deletions, as long as the deletion does not affect the biological activity of hGH, i.e. binding to and initiating signaling through the hGH receptor. A fragment that is preferred according to the invention lacks 15 amino acids from Glutamic acid (Glu) 32 to Glutamic acid 46.

hGH fragments may further be truncated at the C- or N-terminus. Truncated hGH lacking the first eight N-terminal residues or the first 13 N-terminal residues of human growth hormone are also preferred in accordance with the present invention.

A short C-terminal hGH fragment had been described to retain a biological activity of hGH, see U.S. Pat. No. 5,869,452. Therefore, the use of a C-terminal fragment of hGH is preferred according to the invention. Fragment hGH177-191, comprising at least amino acid residues 177 to 191 of hGH (LRIVQCRSVEGSCGF) is particularly preferred in accordance with the present invention. Further preferred are derivatives of this peptide, such as the peptide variants described in U.S. Pat. No. 6,335,319 or W099/12969, e.g. cyclic peptides.

Additionally, the polypeptide, which has such hGH receptor agonist activity, be it hGH, an analog or variant, salt, functional derivative or fragment thereof, can also contain additional amino acid residues flanking the hGH polypeptide. As long as the resultant molecule retains the hGH receptor agonist ability of the core polypeptide, one can determine whether any such flanking residues affect the basic and novel characteristics of the core peptide, i.e., its receptor agonist characteristics, by routine experimentation.

An example for such a GH variant, which is preferred in accordance with the present invention, is methionyl human growth hormone (Met-hGH), which has an additional methionine residue at the N-terminus of human growth hormone.

Variants of hGH, which are preferred according to the invention, comprise methionyl hGH, which is a human growth hormone having an additional methionine residue at its N-terminus. A further preferred variant is a human growth hormone lacking 15 amino acid residues from Glu32 to Glu46.

A “variant” of the human growth hormone according to the present invention refers to a molecule, which is substantially similar to either the entire protein or a fragment thereof. A variant may also be called a “mutein”. A variant may e.g. be an isoform of hGH, such as a variant generated by alternative splicing. Variant (poly)peptides may also be conveniently prepared by direct chemical synthesis of the variant peptide, using methods well known in the art. Of course, a variant human growth hormone would have at least similar hGH receptor binding and signal initiating activity as hGH and which would, therefore, be expected to have similar anti-MSA activity to hGH.

Amino acid sequence variants of the human growth hormone can be prepared by mutations in the DNAs, which encode the synthesized human growth hormone derivatives. Such variants include, for example, deletions from, or insertions or substitutions of, residues within the amino acid sequence. Any combination of deletion, insertion, and substitution may also be made to arrive at the final construct, provided that the final construct possesses the desired activity. Obviously, the mutations that will be made in the DNA encoding the variant peptide must not alter the reading frame.

At the genetic level, these variants may be prepared by site-directed mutagenesis (as exemplified by Adelman et al, 1983) of nucleotides in the DNA encoding the peptide molecule, thereby producing DNA encoding the variant, and thereafter expressing the DNA in recombinant cell culture. The variants typically exhibit at least the same qualitative biological activity as the non-variant peptide.

An “analog” of human growth hormone according to the present invention refers to a non-natural molecule, which is substantially similar to either the entire molecule or to an active fragment thereof. An analog of human growth hormone useful in the present invention would exhibit anti-MSA activity.

The types of substitutions which may be made in the human growth hormone according to the present invention may be based on analysis of the frequencies of amino acid changes between a homologous protein of different species. Based upon such analysis, conservative substitutions may be defined herein as exchanges within one of the following five groups:
I. Small, aliphatic, nonpolar or slightly polar residues: Ala, Ser, Thr, Pro, Gly
II. Polar, negatively-charged residues and their amides: Asp, Asn, Glu, Gln
III. Polar, positvely-charged residues: His, Arg, Lys
IV. Large, aliphatic non-polar residues: Met, Leu, lie,.Val, Cys
V. Large aromatic residues: Phe, Try, Trp

Within the foregoing groups, the following substitutions are considered to be “highly conservative”:
Asp/Glu
His/Arg/Lys
Phe/Tyr/Trp
Met/Leu/lle/Val

Semi-conservative substitutions are defined to be exchanges between two of groups (I)-(IV) above which are limited to supergroup (A), comprising (I), (II), and (III) above, or to supergroup (B), comprising (IV) and (V) above. Substitutions are not limited to the genetically encoded or even the naturally-occurring amino acids. When the epitope is prepared by peptide synthesis, the desired amino acid may be used directly. Alternatively, a genetically encoded amino acid may be modified by reacting it with an organic derivatizing agent that is capable of reacting with selected side chains or terminal residues.

Cysteinyl residues most commonly are reacted with alpha-haloacetates (and corresponding amines), such as chloroacetic acid or chloroacetamide, to give carboxylmethyl or carboxyamidomethyl derivatives. Cysteinyl residues also are derivatized by reaction with bromotrifluoroacetone, alpha-bromo-beta-(5-imidazoyl) propionic acid, chloroacetyl phosphate, N- alkylmaleimides, 3-nitro-2-pyridyl disulfide, methyl-2-pyridyl disulfide, p-chloromercuribenzoate, 2-chloromercuri4-nitrophenol, or chloro-7-nitrobenzo-2-oxa-1,3-diazole.

Histidyl residues are derivatized by reaction with diethylprocarbonate at pH 5.5-7.0 because this agent is relatively specific for the histidyl side chain. Parabromophenacyl bromide is also useful; the reaction is preferably performed in 0.1 M sodium cacodylate at pH 6.0.

Lysinyl and amino terminal residues are reacted with succinic or other carboxylic acid anhydrides. Derivatization with these agents has the effect of reversing the charge of the lysinyl residues. Other suitable reagents for derivatizing alpha-amino acid-containing residues include imidoesters such as methyl picolinimidate; pyridoxal phosphate; pyridoxal; chloroborohydride; trinitrobenzenesulfonic acid; O-methyliosurea; 2,4-pentanedione; and transaminase-catalyzed reaction with glyoxylate.

Arginyl residues are modified by reaction with one or several conventional reagents, among them phenylglyoxal; 2,3- butanedione; and ninhydrin. Derivatization of arginine residues requires that the reaction be performed in alkaline conditions because of the high pKa of the guanidine functional group. Furthermore, these reagents may react with the groups of lysine, as well as the arginine epsilon-amino group.

The specific modification of tyrosyl residues perse has been studied extensively, with particular interest in introducing spectral labels into tyrosyl residues by reaction with aromatic diazonium compounds or tetranitromethane. Most commonly, N-acetylimidazole and tetranitromethane are used to form O-acetyl tyrosyl species and ε-nitroderivatives, respectively.

Carboxyl side groups (aspartyl or glutamyl) are selectively modified by reaction with carbodiimides (R′N—C—N—R′) such as 1-cyclohexyl-3-[2-morpholinyl-(4-ethyl)]carbodiimide or 1-ethyl-3-(4-azonia-4,4-dimethylpentyl)carbodiimide. Furthermore, aspartyl and glutamyl residues are converted to asparaginyl and glutaminyl residues by reaction with ammoniumions.

Glutaminyl and asparaginyl residues are frequently deamidated to the corresponding glutamyl and aspartyl residues. Alternatively, these residues are deamidated under mildly acidic conditions. Either form of these residues falls within the scope of this invention.

Examples of production of amino acid substitutions in proteins which can be used for obtaining analogs of the hGH for use in the present invention include any known method steps, such as presented in U.S. Pat. No. RE 33,653; U.S. Pat. Nos. 4,959,314; 4,588,585 and 4,737,462, to Mark et al; U.S. Pat. No. 5,116,943 to Koths et al; U.S. Pat. No. 4,965,195 to Namen et al; and U.S. Pat. No. 5,017,691 to Lee, et al, and lysine substituted proteins presented in U.S. Pat. No. 4,904,584 (Shaw et al). Further growth hormone variants have been described e.g. in U.S. Pat. No. 6,143,523 (Cunningham et al.).

Among the substances which bind to and initiate signaling of the human growth hormone receptor which may be used in accordance with the present invention are all of those growth hormone analogs and mimetics already known in the literature, such as, for example, those disclosed in U.S. Pat. Nos. 5,851,992; 5,849,704; 5,849,700; 5,849,535; 5,843,453; 5,834,598; 5,688,666; 5,654,010; 5,635,604; 5,633,352; 5,597,709; and 5,534,617.

Preferably, the hGH variant or analog will have a core sequence, which is the same as that of the native sequence or biologically active fragment thereof, which has an amino acid sequence having at least 70% identity to the native amino acid sequence and retains the biological activity thereof. More preferably, such a sequence has at least 80% identity, at least 90% identity, or most preferably at least 95% identity to the native sequence.

“Identity” reflects a relationship between two or more polypeptide sequences or two or more polynucleotide sequences, determined by comparing the sequences. In general, identity refers to an exact nucleotide to nucleotide or amino acid to amino acid correspondence of the two polynucleotides or two polypeptide sequences, respectively, over the length of the sequences being compared.

For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

Methods for comparing the identity and homology of two or more sequences are well known in the art. Thus for instance, programs available in the Wisconsin Sequence Analysis Package, version 9.1 (Devereux J et al., 1984), for example the programs BESTFIT and GAP, may be used to determine the % identity between two polynucleotides and the % identity and the % homology between two polypeptide sequences. BESTFIT uses the “local homology” algorithm of Smith and Waterman (1981) and finds the. best single region of similarity between two sequences. Other programs for determining identity and/or similarity between sequences are also known in the art, for instance the BLAST family of programs (Altschul S F et al, 1990, Altschul S F et al, 1997, accessible through the home page of the NCBI at www.ncbi.nlm.nih.gov) and FASTA (Pearson W R, 1990; Pearson 1988).

Preferred changes for variants or muteins in accordance with the present invention are what are known as “conservative” substitutions. Conservative amino acid substitutions of growth hormone polypeptides or proteins, may include synonymous amino acids within a group which have sufficiently similar physicochemical properties that substitution between members of the group will preserve the biological function of the molecule (Grantham, 1974). It is clear that insertions and deletions of amino acids may also be made in the above-defined sequences without altering their function, particularly if the insertions or deletions only involve a few amino acids, e.g., under thirty, and preferably under ten, and do not remove or displace amino acids which are critical to a functional conformation, e.g., cysteine residues. Proteins and muteins produced by such deletions and/or insertions come within the purview of the present invention.

Analogs or variants in accordance with the present invention may also be determined in accordance with the following procedure. The DNA of the native sequence is known to the prior art and is found in the literature (Martial et al, 1979). Polypeptides encoded by any nucleic acid, such as DNA or RNA, which hybridizes to the complement of the native DNA or RNA under highly stringent or moderately stringent conditions, as long as that polypeptide maintains the biological activity of the native sequence, are also considered to be within the scope of the present invention.

Stringency conditions are a function of the temperature used in the hybridization experiment, the molarity of the monovalent cations and the percentage of formamide in the hybridization solution. To determine the degree of stringency involved with any given set of conditions, one first uses the equation of Meinkoth et al. (1984) for determining the stability of hybrids of 100% identity expressed as melting temperature Tm of the DNA-DNA hybrid:
Tm=81.5° C.+16.6 (_{Log}M)+0.41 (%GC)−0.61 (% form)−500/L
where M is the molarity of monovalent cations, %GC is the percentage of G and C nucleotides in the DNA, % form is the percentage of formamide in the hybridization solution, and L is the length of the hybrid in base pairs. For each 1° C. that the Tm is reduced from that calculated for a 100% identity hybrid, the amount of mismatch permitted is increased by about 1%. Thus, if the Tm used for any given hybridization experiment at the specified salt and formamide concentrations is 10° C. below the Tm calculated for a 100% hybrid according to equation of Meinkoth, hybridization will occur even if there is up to about 10% mismatch.

As used herein, highly stringent conditions are those which are tolerant of up to about 15% sequence divergence, while moderately stringent conditions are those which are tolerant of up to about 20% sequence divergence. Without limitation, examples of highly stringent (12-15° C. below the calculated Tm of the hybrid) and moderately (15-20° C below the calculated Tm of the hybrid) conditions use a wash solution of 2×SSC (standard saline citrate) and 0.5% SDS at the appropriate temperature below the calculated Tm of the hybrid. The ultimate stringency of the conditions is primarily due to the washing conditions, particularly if the hybridization conditions used are those, which allow less stable hybrids to form along with stable hybrids. The wash conditions at higher stringency then remove the less stable hybrids. A common hybridization condition that can be used with the highly stringent to moderately stringent wash conditions described above is hybridization in a solution of 6×SSC (or 6×SSPE), 5× Denhardt's reagent, 0.5% SDS, 100 μg/ml denatured, fragmented salmon sperm DNA at a temperature approximately 20° to 25° C. below the Tm. If mixed probes are used, it is preferable to use tetramethyl ammonium chloride (TMAC) instead of SSC (Ausubel, 1987-1998).

While the present invention provides recombinant methods for making the human growth hormone derivatives, these derivatives may also be made by conventional protein synthesis methods which are well known to those skilled in the art.

Human growth hormone, or fragments, variants, analogs, or functional derivatives, or salts thereof may be administered at various dosages.

In a preferred embodiment, growth hormone is administered at a dosage of about 0.1 to 10 mg per person per day or about 0.5 to 6 mg per person per day.

In a further preferred embodiment, the growth hormone is administered at a dosage of about 1 mg per person per day.

In yet a further preferred embodiment, the growth hormone is administered daily or every other day.

In accordance with the present invention, growth hormone may also be administered at alternating daily dosages, the first dosage being higher than the second dosage. Preferably, the first dosage is about 1 mg per person and the second dosage is about 0.5 mg per person.

In another preferred embodiment of the invention, the weekly dosage of growth hormone is about 6 mg per person or about 5 mg per person or about 4.5 mg per person.

The growth hormone treatment in accordance with the present invention may be accomplished either by administration of exogenous growth hormone or by administration of a substance which stimulates production of endogenous growth hormone either directly or indirectly by suppressing endogenous somatostatin secretion.

Therefore, in a further preferred embodiment of the present invention the substance which binds to and initiates signaling of the human growth hormone (hGH) receptor or a substance which stimulates release or potentiates the activity of endogenous hGH, which is used for the preparation of a medicament for treatment and/or prevention of a Parkinsonism-Plus Syndrome, in particular Multiple System Atrophy, is selected from:
(a) a human growth hormone releasing hormone (hGHRH);
(b) a fragment of (a) which has agonistic activity on the hGHRH receptor;
(c) a variant of (a) or (b) which has at least 70% sequence identity with (a) or (b) and which has agonistic activity on the hGHRH receptor;
(d) a variant of (a) or (b) which is encoded by a DNA sequence which hybridizes to the complement of the native DNA sequence encoding (a) or (b) under moderately stringent conditions and which has agonistic activity on the hGHRH receptor; or
(e) a salt or functional derivative of (a), (b), (c) or (d) which has agonistic activity on the hGHRH receptor.

It is known that human growth hormone releasing hormone (hGHRH) stimulates the release of hGH. Thus, the biological activity of hGH can be indirectly obtained by administering GHRH or a functional derivative, salt, variant, analog or fragment thereof which retains the biological activity of GHRH, i.e., the ability to stimulate the release of growth hormone. Thus, for example, besides GHRH there may be used functional derivatives thereof in accordance with the above definition, analogs or variants thereof, which have at least 70% sequence identity, more preferably 80% or 90% or, most preferably, 95% sequence identity therewith, yet retains the biological activity of GHRH, or a variant or analog which is a polypeptide encoded by a DNA which hybridizes to the native DNA encoding GHRH under moderately stringent conditions, or preferably under highly stringent conditions, all in accordance with the definitions given hereinabove.

In a preferred embodiment of the present invention, functional derivatives of hGH or GHRH, or any active fragment, variant, or analogue thereof, comprises at least one moiety attached to one or more functional groups, which occur as one or more side-chains on the amino acid residues. The attachment of polyethylene glycol (PEG) is preferred. Preferred PEGylated GHRH (also called GRF) molecules, which can be used in connection with the present invention, have been described in W099/27897, for example. The substances of the present invention may also be alkylated in order to prolong the half-life within the human body.

Long-lasting formulations, such as formulations in which the half-life (T_{H}) of the active substances is higher than 30 hours, are particularly preferred in accordance with the present invention.

Any of the GHRH or GHRH analogs or agonists known in the literature and disclosed as simulating the release of growth hormone may also be used in the present invention, such as those disclosed in U.S. Pat. Nos. 5,792,747; 5,776,901; 5,696,089; 5,137,872; 5,767,085; 5,612,470; 5,846,936; and 5,847,066. See also Thorner et al (1997), Felix et al (1995), Alba-Roth et al (1988), Friend et al (1997).

Other substances capable of promoting the release of growth hormone in vivo which can be used in accordance with the present invention include those disclosed in U.S. Pat. Nos. 5,807,985; 5,804,578; 5,795,957; 5,777,112; 5,767,118; 5,731,317; 5,726,319; 5,726,307; 5,721,251; 5,721,250, etc.

Any other molecule, which binds to the hGH receptor and initiates signaling of that receptor may also be used in accordance with the present invention. It is known, for example, that small molecules, sometimes called secretagogues, have been developed which bind hGH receptors and cause them to aggregate and initiate signaling, which signal initiation is the same as one obtains with natural hGH binding to the receptor. Such molecules are known, for example, from U.S. Pat. Nos. 5,773,441; 5,798,337; 5,830,433; 5,767,124; and 5,723,616. See also Bowers et al (1991), Thorner et al (1997), Camanni et al (1998), Smith et al (1993) and Ghigo et al (1998).

Thus, the present invention is intended to include any substance, which binds to hGH receptor and initiates signaling thereof so as to obtain the same ultimate qualitative effect as the administration of natural hGH, insofar as the treatment of Parkinsonism-Plus Syndromes, in particular MSA is concerned.

It is well known in the art that insulin-like growth factors (IGFs) belong to the signaling cascade of growth hormone. Two IGFs have been described so far, called IGF-I and IGF-II. IGF-I mediates most of the growth-promoting actions of GH. IGF-I, a potent mitogenic growth factor, bears a striking homology to proinsulin. It binds to specific receptors that also bind insulin at lower affinity. The predominant site of GH-stimulated IGF-I production is the liver, whereas several extra-hepatic tissues also synthesize IGF-I. IGF-I regulates GH gene expression and secretion by a negative feedback regulation, analogous to the inhibition by thyroid and adrenal hormones of their respective pituitary trophic hormones.

Therefore, the invention further relates to the use of an IGF (Insulin-like Growth Factor) for the preparation of a medicament for treatment and/or prevention of a Parkinsonism-Plus Syndrome, in particular Multiple System Atrophy. Preferably, the IGF is selected from IGF-I, or IGF-II.

It is also known that IGFs form complexes with specific binding proteins, called IGF binding proteins (IGFBPs). These binding proteins have been proposed to perform four functions including transporting IGFs in the vasculature and a cross intact capillary membranes, localizing the IGFs to specific tissues and cell types, controlling IGF interaction with cell surface receptors and modulating biologic actions of the IGFs. (Clemmons et al., 1993) In particular, Insulin-like growth factor (IGF)-binding protein-3 (IGFBP-3) is a major determinant of circulating levels of the IGFs and is clinically useful for the evaluation of GH deficiency and for predicting the response to GH treatment.

Therefore, in a preferred embodiment of the invention, the medicament further comprises an IGFBP. Preferably, the IGFBP is IGFBP3.

Combinations of IGF-I, IGF-II, an IGFBP with human growth hormone or GHRH, or any of their fragments, variants, functional derivatives or salts, for treatment and/or prevention of Parkinson-Plus syndrome, in particular MSA, are further within the present invention. The substances may be used sequentially, separately or simultaneously.

The invention further relates to the use of an nucleic acid molecule comprising the coding sequence of a substance which binds to and initiates signaling of the human growth hormone (hGH) receptor, or a substance which stimulates release or potentiates the activity of endogenous hGH, for the preparation of a medicament for the treatment and/or prevention o f a Parkinsonism-Plus Syndrome, in particular Multiple System Atrophy.

The nucleic acid molecule may further comprise a sequence of an expression vector, e.g. to use gene therapy for administering the hGH in accordance with the invention.

Preferably, the medicament of the invention is administered subcutaneously.

It is also preferred to administer the medicament intramuscularly.

In yet another preferred embodiment, the substance is administered with an auto-injector. Auto-injectors are devices facilitating subcutaneous administration of medicaments. Auto-injectors are known in the art, such as the one called Easyject®, which is particularly useful for administration of hGH. Needle-free administration may also be used in connection with the present invention, using special devices that are known in the art.

The invention further relates to the use of a vector for inducing and/or enhancing the endogenous production of a substance, which binds to and initiates signaling of the human growth hormone (hGH) receptor or a substance which stimulates release or potentiates the activity of endogenous hGH for the preparation of a medicament for the treatment and/or prevention of a Parkinsonism-Plus Syndrome, in particular Multiple System Atrophy.

The vector may comprise regulatory elements functional in the cells desired to express the substance of the invention. Such regulatory sequences or elements may be promoters or enhancers, for example. The regulatory sequence may then be introduced into the right locus of the genome by homologous recombination, thus operably linking the regulatory sequence with the gene, the expression of which is required to be induced or enhanced. The technology is usually referred to as “Endogenous Gene Activation” (EGA), and it is described e.g. in WO 91/09955.

The invention further relates to the use of a cell that has been genetically modified to produce a substance which binds to and initiates signaling of the. human growth hormone (hGH) receptor or a substance which stimulates release or potentiates the activity of endogenous hGH for the preparation of a medicament for the treatment and/or prevention of a Parkinsonism-Plus Syndrome, in particular Multiple System Atrophy.

The invention further relates to a method for treating a Parkinsonism-Plus Syndrome, in particular Multiple System Atrophy, comprising administering to a patient in need thereof an effective amount of a substance which binds to and initiates signaling of the human growth hormone (hGH) receptor or a substance which stimulates release or potentiates the activity of endogenous hGH.

Pharmaceutical compositions for administration according to the present invention can comprise at least one human growth hormone according to the present invention in a pharmaceutically acceptable form, optionally combined with a pharmaceutically acceptable carrier, excipient, stabilizer or auxiliary agent.

These compositions can be administered by any means that achieve their intended purposes. Amounts and regimens for the administration of a composition according to the present invention can be determined readily by those with ordinary skill in the art for treating Parkinsonism-Plus Syndromes, in particular MSA.

Compositions within the scope of this invention include all composition comprising at least one human growth hormone or derivative, analog, or variant thereof according to the present invention in an amount effective to achieve its intended purpose. While individual needs vary, determination of optimal ranges of effective amounts of each component is within the skill of the art. Typical dosages comprise about 0.001 to about 0.1 mg/kg body weight per day. When administered to MSA patients, the hGH anti-MSA therapy may be administered concomitantly with other therapies which may be indicated in this disease.

In a preferred embodiment of the invention, hGH is administered in a daily dosage of about 0.1 to 10 mg or about 0.5 to 6 mg. Further preferred is a dosage of about 1 mg of human growth hormone per person per day.

In a further preferred embodiment, hGH is administered at alternating dosages, the first dosage being higher than the second dosage. Preferably, the first dosage is about 1 mg and the second dosage is about 0.5 mg. Weekly dosages are preferably about 6 mg or about 5 mg or about 4.5 mg, depending on the needs of the patient.

For example, administration can be by parenteral, such as subcutaneous, intravenous, intramuscular, oral, intraperitoneal, aerosol, transdermal, intrathecal, or rectal routes. The dosage administered depends upon the age, health and weight of the recipient, type of previous or concurrent treatment, if any, frequency of the treatment and the nature of the effect desired.

In accordance with the present invention, preferred administration routes are the subcutaneous and the intramuscular routes. An especially preferred route of administration is the oral route.

It should also be understood that, to be useful, the treatment provided need not be absolute, provided that it is sufficient to carry clinical value. An agent which provides treatment to a lesser degree than do competitive agents may still be of value if the other agents are ineffective for a particular individual, if it can be used in combination with other agents to enhance the overall level of protection, or if it is safer than competitive agents.

It is understood that the suitable dose of a composition according to the present invention will depend upon the age, health and weight of the recipient, kind of concurrent treatment, if any, frequency of treatment, and the nature of the effect desired. However, the most preferred dosage can be tailored to the individual subject, as is understood and determinable by one of skill in the art, without undue experimentation. This typically involves adjustment of a standard dose, e.g., reduction of the dose if the patient has a low body weight.

The total dose required for each treatment may be administered in multiple doses or in a single dose. The compositions may be administered alone or in conjunction with other therapeutics directed to the disease or directed to other symptoms thereof.

In addition to the compounds of the invention, a pharmaceutical composition may contain suitable pharmaceutically acceptable carriers, such as excipients, carriers and/or auxiliaries, which facilitate processing of the active compounds into preparations which can be used pharmaceutically.

Having now fully described this invention, it will be appreciated by those skilled in the art that the same can be performed within a wide range of equivalent parameters, concentrations and conditions without departing from the spirit and scope of the invention and without undue experimentation.

While this invention has been described in connection with specific embodiments thereof, it will be understood that it is capable of further modifications. This application is intended to cover any variations, uses or adaptations of the invention following, in general, the principles of the invention and including such departures from the present disclosure as come within known or customary practice within the art to which the invention pertains and as may be applied to the essential features hereinbefore set forth as follows in the scope of the appended claims. All references cited herein, including journal articles or abstracts, published or unpublished U.S. or foreign patent application, issued U.S. or foreign patents or any other references, are entirely incorporated by reference herein, including all data, tables, figures and text presented in the cited references. Additionally, the entire contents of the references cited within the references cited herein are also entirely incorporated by reference.

Reference to known method steps, conventional methods steps, known methods or conventional methods is not any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning an range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

Having now described the invention, it will be more readily understood by reference to the following example of an exemplary clinical study outline, that is provided by way of illustration, and not intended to be limiting of the present invention.

### EXAMPLE

### List of Abbreviations

AE Adverse event
AST Aspartate Transaminase
ALT Alanine Transaminase
CNS Central Nervous System
CPMP Committee for Proprietary Medicinal Products
CRA Clinical Research Associate
CRF Case Report Form
CSF Cerebrospinal Fluid
DER Drug Event Report (form)
FDA Food and Drug Administration (US)
GCP Good Clinical Practice
GFAP Glial Fibrillary Acid Protein
GH Growth Hormone
GHD Growth Hormone Deficiency
GLP Good Laboratory Practice
hCG Human Chorionic Gonadotrophin
5-HIAA 5-Hydroxyindoleacetic acid
HVA Homovanillic acid
ICH International Conference on Harmonisation
IEC Independent Ethics Committee
IGF-1 Insulin-like Growth Factor I
IGFBP-3 Insulin-like Growth Factor binding protein 3
IRB Institutional Review Board
IU International Unit
IV Intravenous
L-DOPA Levodopa
LTP Long term potentiation
MG Milligram
MRA Medical Research Associate
MSA Multiple System Atrophy
NFL Neurofilament protein
NHP Nottingham Health Profile
PLM Posturo-Locomotor Manual test
r-hGH Recombinant Human Growth Hormone
RNA Ribonucleic Acid
SAE Serious adverse event
TD Therapeutic Director
TPN Total parenteral nutrition
UPDRS Unified Parkinson's Disease Rating Scale
WHO World Health Organisation

Study Synopsis
Title: A phase 11, single center, double-blind, randomized, placebo-controlled study of subcutaneously administered recombinant human growth hormone (r-hGH) in the treatment of Multiple System Atrophy (MSA)
Proiect phase: Phase II
Indication: Multiple System Atrophy
   Primary Objectives:
1) Laboratory: To determine the effect of treatment with r-hGH by the analysis of Glial Fibrillary Acid Protein (GFAP) and Neurofilament Protein (NFL), markers for neurodegeneration, in the cerebrospinal fluid (Holmberg et al., 1998).
2) Clinical:
(a) Stabilization as compared with placebo group in functional assessments measured with the Unified Parkinson's Disease Rating Scale (see below).
(b) Stabilization as compared with placebo in results of autonomic testing
   Secondary Objectives:
(a) To assess the safety and tolerability of r-hGH in this patient population
(b) To detect any difference between groups in disease progression using the Posturo-Locomotor-Manual test (PLM)
(c) Improvement in quality of life as measured using the Nottingham Health Profile (NHP) (see below)

Sample size: 40 evaluable patients

Study drug: Saizen® [recombinant human Growth Hormnone (r-hGH)]

Treatment regimen: 1 mg three times per week for 12 months (with possible dose escalation at 6 months to alternating daily injections of 1 mg and 0.5mg, if the patient is significantly worse).

Route of administration: subcutaneous

Procedures: Patients will be required to fulfil the criteria for a clinically probable diagnosis of MSA before inclusion. There will be 6 visits, a pre-study evaluation, study day 1, month 3, 6, 9 and 12. Analyses of cerebrospinal fluid for GFAP and NFL will take place at pre-study evaluation, month 6 and month 12. At study day 1, month 6 and month 12, the patient will be asked to complete the NHP questionnaire, they will be assessed regarding functionality with the UPDRS and autonomic testing will be performed.

Safety and efficacy data will be obtained through routine clinical follow-up, routine haematology, clinical chemistry and urinalysis, in addition to the specific procedures mentioned below.

Obiectives

The objectives of the study are:

Primary Objectives

Laboratory: To determine the effect of treatment with r-hGH by the analysis of GFAP and NFL (markers for neurodegeneration) in the cerebrospinal fluid (Holmberg et al., 1998) i.e. the stabilisation of GFAP and NFL in the patients receiving r-hGH and an increase in these markers, as a sign of continuing degradation, in the placebo group.
Clinical:
Stabilization as compared with the placebo group in functional assessments as measured with the Unified Parkinson's Disease Rating Scale (Appendix F).
Stabilization as compared with placebo in results of autonomic testing (Appendix I).
   Secondary Objectives
To assess the safety and tolerability of r-hGH in this patient population.
To detect any difference between groups in disease progression using the Posturo-Locomotor Manual test (PLM) (see below).
Improvement in quality of life as measured using the Nottingham Health Profile (NHP) (see below).
   Study Population

Forty patients will be included in the study. Each patient must:
Meet all of the inclusion and exclusion criteria specified in the following sections within the specified time frame,
Receive the allotted course of treatment and complete the required activities specified in the protocol, and
Have his or her Case Report Form (CRF) completed, received and have all queries resolved to the standard required by the Sponsor.
   Inclusion Criteria

To be eligible for inclusion into this study, each patient must fulfil the following criteria within 28 days prior to Study Day 1:
1) Fulfil the criteria for a clinically probable diagnosis of MSA (below)
2) Have a life expectancy of at least one year
3) Should be between 30 and 75 years of age
4) Willingness and ability to comply with the protocol for the duration of the study.
5) Written informed consent given prior to any study-related procedure not part of the patient's normal medical care, with the understanding that the patient may withdraw consent at any time without prejudice to future medical care.
6) Female patients must
Either (a) Be post-menopausal or surgically sterilised
Or (b) Use a hormonal contraceptive, intra-uterine device, diaphragm with spermicide or condom with spermicide for the duration of the study,
And (c) Must be neither pregnant nor breast-feeding.

Confirmation that a female patient is not pregnant must be established by a negative serum/urinary hCG pregnancy test during the 28 day screening period prior to Study Day 1. A pregnancy test is not required if the patient is post-menopausal or surgically sterilised.

Exclusion Criteria

Patients meeting any of the following criteria will be excluded from the study:
1. Clinical evidence of concomitant infection or inflammatory disease in the blood or cerebrospinal fluid.
2. Serum creatinine, AST or ALT >2.5×the upper value of the normal range (values not to be older than 1 month prior to Study Day 1).
3. Presence or history of diabetes mellitus (type I or II).
4. Presence or history of any active malignancy.
5. Hypothyroidism (unless adequately treated with thyroid hormone replacement therapy).
6. Benign cranial hypertension.
7. Previous history of Carpal Tunnel Syndrome not surgically released.
8. Patients who are poor medical risks because of non-malignant organ or systemic disease or significant secondary effects of cancer are not eligible. Patients with clinically significant cardiac disease (i.e. presence of defined cardiac symptoms with marked limitations and need for additional rest to control symptoms) are not eligible.
9. Have taken another investigational drug or have taken part in any experimental procedure in the 3 months preceding study entry.
10. History of prior allergy to r-hGH.
11. Previous treatment with r-hGH.
   Assignment of Patient Numbers and Treatment Groups

Forty patients will be enrolled into the study and on entry, will be randomised to receive subcutaneous injections of either SAIZEN® or placebo 1 mg, in a double-blind manner for 12 months (with possible dosees calation at 6 months to alternating daily injections of lmg and 0.5mg, if the patient is significantly worse). The treatment assigned to each patient will be determined according to a computer-generated randomisation list. The patient packs and enclosed vials will be labelled with a unique patient identification number.

When a patient has been found eligible for the study and completed all the pre-study procedures, he/she will be allocated a unique patient identification number in sequential, chronological order after completion of all the baseline assessments on Study Day 1. Prior to allocation of a patient number, all patients should be identified by their initials and date of birth. Should a patient be withdrawn from the study after randomization, his or her identification number will not be reallocated.

Study Medication

Presentation, Preparation, Storage and Labelling

The study drug, (Saizen® or placebo), will be supplied by Serono as a multidose preparation in glass vials containing 24IU (8.8 mg) r-hGH plus excipients (sucrose, phosphoric acid 85% and phosphoric acid diluted from phosphoric acid 85% or sodium hydroxide) or matching placebo glass vials containing excipients only. The solvent for reconstitution will be supplied in cartridges containing Metacresol 0.3% (w/v) in water for injection.

One vial of study drug will be reconstituted with 1.51 ml diluent. The Easyject® Auto-Injector will be used for this study and will be supplied to patients along with the reconstitution kits and needles.

The lyophilised product is to be stored at or below 25° C. and protected from light. All study drugs must be stored in a secure location, preferably in a locked, temperature-controlled refrigerator or cold room. Study drugs may be dispensed only by the Investigator, by a pharmacist or by a member of staff specifically authorised by the Investigator, as appropriate. Any deviations from the recommended storage conditions should be reported to the Sponsor immediately, and use of the study drug should be interrupted until the Sponsor has authorised its continued use.

Once reconstituted with bacteriostatic diluent, the drug should be stored between 2° C. and 8° C. (36° F. and 46° F.) and used within 21 days for injection Labelling and packaging will be prepared to meet local regulatory requirements.

Dose, Route and Schedule of Study Drug Administration

Each patient will receive a subcutaneous injection preferably at bedtime, three times a week (preferably Monday, Wednesday and Friday) at a dose of 1 mg for 12 months. However, if at the 6-month visit the patient is significantly worse in any of the following clinical symptoms or findings the dose will be escalated to alternating daily injections of 1 mg and 0.5 mg.
Worsening of dysarthria, dysphagia, paresis, coordination, rigidity, balance, walking ability, urinary continence or sexual function
Increased levels of NFL in the cerebrospinal fluid
Further impairment of results of the cardiovascular reflex tests

Injection sites should be rotated on the abdomen, arms and legs. At the Investigator's discretion, the patient or a family member/partner may be taught to administer the injections.

The patient will be asked to keep a record of the time of injection, the amount injected and any local or systemic adverse events on a diary card.

Concomitant Therapy

The use of anti-coagulant medication (with the exception of Aspirin) during the study is not allowed.

With the exceptions noted, any medications that are considered necessary for the patient's welfare and that will not interfere with the study medication may be given at the Investigator's discretion. Administration of all concomitant drugs must be reported in the appropriate section of the CRF along with dosage information, route, dates of administration and reasons for use. Additionally, any un planned diagnostic, therapeutic or surgical procedures performed during the study period must be recorded in the concomitant procedure section of the CRF, including the date, indication and description of the procedures and their outcomes.

The use of any herbal/natural products or other “folk remedies”, vitamins, nutritional supplements and all other concomitant medications must be recorded in the case report form in the same way as conventional drugs.

As this is an outpatient study, patients will be asked to record details of any self-medication on a diary card.

Informed Consent

Each potentially eligible patient will be informed of the study's objectives and overall requirements. Before conducting any of the pre-entry tests not performed routinely in the patient's treatment, the Investigator will explain the study fully to the patient using the Patient Information Leaflet/Informed Consent Form. If the patient is willing to participate in the study, written informed consent will be requested after the patient has been given sufficient time to consider participation and the opportunity to ask for further details. The Informed Consent Form will be signed and personally dated by both the patient and the Investigator/Sub-investigator. A copy of the signed form will be provided to the patient, and the original will be retained with the source documents. Although nursing staff may be involved in describing the trial to a patient, the Investigator/Sub-investigator must participate in discussions with the patient and must sign and personally date the Informed Consent Form.

A short CRF will be completed for all patients who sign the Informed Consent Form but who do not subsequently enter the study. These patients will be identified by their initials and dates of birth; in addition, their race, sex and reasons for exclusion from the study will be recorded.

Pre-Study Evaluation

Within 28 days of Study Day 1 (day of first injection of r-hGH), patients must be evaluated to determine if they are eligible for the study. This evaluation should involve:
Medical review and history sufficient to satisfy Inclusion and Exclusion criteria (a lab result should be available in order to satisfy the exclusion criterion 2, i.e. creatinine, AST/ALT).
A lumbar puncture will be performed and a sample of cerebrospinal fluid will be taken. The sample will be sent to the local laboratory for the analysis of GFAP and NFL levels.
   Study Day 1

The following baseline data and assessments should be collected/performed and recorded on Study Day 1 prior to the first injection of r-hGH. These include:
Complete medical history unrelated to MSA.
History of the condition under study, including date of diagnosis, sub-type if known and previous therapy
Collection of demographic data, including dates of birth, ethnicity and gender.
Physical examination, including body weight and vital signs.
Current disease related medical conditions, concomitant medications and procedures
Routine haematology, clinical chemistry, endocrinology, urinalysis and antibodies to hGH
The patient will be requested to complete the Nottingham Health Profile quality of life questionnaire (see below). The Investigator or study nurse will explain this to the patient.
The Investigator will assess the patient's functional ability by completing the Unified Parkinson's Disease Rating Scale (UPDRS) (see below)
The Posturo-Locomotor Manual (PLM) Test will be performed by the Investigator as described below.
Autonomic testing will be performed by the Investigator (heart rate variability at controlled forced respiration, blood pressure and heart rate responses to tilt)
   Month 3

The following procedures will be performed at the end of Month 3 (no more than a week earlier or a week later than the scheduled visit):
Physical examination including vital signs
Assessment of adverse events, concomitant medications and procedures
Routine haematology, clinical chemistry and urinalysis
Month6

The following procedures will be performed at the end of Month 6 (no more than a week earlier or a week later than the scheduled visit):
Physical examination including vital signs
Routine haematology, clinical chemistry, endocrinology, urinalysis and antibodies to r-hGH.
Assessment of adverse events, concomitant medications and procedures
A lumbar puncture will be performed and a sample of cerebrospinal fluid will be taken. The sample will be sent to the local laboratory for the analysis of GFAP and NFL levels.
The patient will be requested to complete the Nottingham Health Profile quality of life questionnaire. This will be explained to the patient by the Investigator or study nurse.
The Investigator will assess the patient's functional ability by completing the Unified Parkinson's Disease Rating Scale (UPDRS)
Autonomic testing will be performed by the Investigator (he art rate variability at controlled forced respiration, blood pressure and heart rate responses to tilt) If the patient is significantly worse in any of the following clinical symptoms or findings, the dose will be escalated to alternating daily injections of 1 mg and 0.5 mg.
Worsening of dysarthria, dysphagia, paresis, coordination, rigidity, balance, walking ability, urinary continence or sexual functions
Increased levels of CSF-NFL
Further impairment of results of the cardiovascular reflex tests
   Month 9

The following procedures will be performed at the end of Month 9 (no more than a week earlier or a week later than the scheduled visit):
Physical examination including vital signs
Assessment of adverse events, concomitant medications and procedures
Routine haematology, clinical chemistry and urinalysis
   Month 12 (or Following Withdrawal)

The following procedures will be performed at the end of Month 12 (no more than a week earlier or a week later than the scheduled visit), i.e. the end of treatment with r-hGH, or following early withdrawal of the patient from the study:
Physical examination including vital signs
Routine haematology, clinical chemistry, endocrinology, urinalysis and antibodies to hGH.
Assessment of adverse events, concomitant medications and procedures
A lumbar puncture will be performed and a sample of cerebrospinal fluid will be taken. The sample will be sent to the local laboratory for the analysis of GFAP and NFL levels.
The patient will be requested to complete the Nottingham Health Profile quality of life questionnaire. This will be explained to the patient by the Investigator or study nurse.
The Investigator will assess the patient's functional ability by completing the Unified Parkinson's Disease Rating Scale (UPDRS)
The Posturo-Locomotor Manual (PLM) Test will be performed.
Autonomic testing will be performed (heart rate variability at controlled forced respiration, blood pressure and heart rate responses to tilt).

In case of an ongoing adverse event, such as a clinically significant laboratory abnormality, appropriate safety evaluations should be repeated more frequently and/or additional tests performed when clinically indicated (or at the Investigator's discretion) until resolution, or until a period of 30 days has elapsed after the last dose of study drug, whichever is the shorter.

Premature Study Discontinuation and Replacement Policy

Discontinuation Criteria

Patients will be informed that they have the right to withdraw from the study at any time, without prejudice to their medical care, and that they are not obliged to state their reasons. Any withdrawal must be fully documented in the CRF, and should be followed up by the Investigator.

Additionally, the Investigator may withdraw a patient at any time if this is considered to be in the patient's best interest.

During the course of the study, the patient must be discontinued for the following reason:
(modified) WHO Grade 3 or 4 toxicity considered by the Investigator to be related to the study drug

Patients may be discontinued for these reasons:
Admission to an Intensive Care Unit.
Protocol violations, including non-compliance and loss to follow-up,
Serious intercurrent illness or significant worsening of intercurrent illness,
Adverse events, or
Administrative reasons.

If a patient fails to return for follow-up, attempts should be made to contact the patient to ensure that the reason for not returning is not an adverse event. Likewise, if a patient decides to discontinue from the study, e.g. for personal reasons, an attempt should be made to establish that the true reason is not an adverse event (bearing in mind that patients are not obliged to state their reasons).

If the study drug therapy is prematurely discontinued, the primary reason for discontinuation must be recorded in the appropriate section of the CRF and all efforts must be made to complete and report the observations as thoroughly as possible. A complete final evaluation should be made following the patient's withdrawal as described in section 6.7. Any adverse events should be followed up until resolution or until a period of 30 days has elapsed after the last dose of study drug, whichever is the shorter.

Replacement Policy

Should a patient drop out or be withdrawn from the study, his or her number will not be reallocated. All ineligible (patients mistakenly included who do not fulfil the eligibility criteria) patients must be replaced as 40 evaluable patients are needed to evaluate the study.

Adverse Event Reporting

Definition

An adverse event (AE) is defined as any untoward medical occurrence in a patient or clinical investigation subject administered a pharmaceutical product, which does not necessarily have a causal relationship with this treatment. An adverse event can therefore be any unfavourable and unintended sign (including an abnormal laboratory finding), symptom, or disease temporally associated with the use of a medicinal (investigational) product, whether or not related to the medicinal (investigational) product.

Reporting

All AEs, as defined above, encountered during the clinical study as well as any serious adverse events, will be reported in the appropriate section of the CRF. It is important that this includes the duration of the AE (onset/resolution dates), the severity, the relationship to the drug (possible, probably, unlikely—see below), the frequency and any concomitant treatment dispensed (or other action taken).

Probable: A clinical event including laboratory test abnormality with a reasonable time sequence to administration of the drug unlikely to be at tributed to concurrent disease or other drugs or chemicals and which follows a clinically reasonable response on withdrawal (dechallenge). Rechallenge information is not required to fulfil this definition.

Possible: A clinical event including laboratory test abnormality with a reasonable time sequence to administration of the drug but which could also be explained by concurrent disease or other drugs or chemicals. Information on drug withdrawal may be lacking or unclear.

Unlikely: A clinical event including laboratory test abnormality with temporal relationship to drug administration which makes a causal relationship improbable and in which other drugs, chemicals or underlying disease provide plausible explanations Severity of adverse events will be graded according to the modified WHO toxicity grading scale. If an AE occurs that is not listed among these criteria, the Investigator will evaluate its severity using the following definitions:

Mild: The patient is aware of the event or symptom, but the event or symptom is easily tolerated.

Moderate: The patient experiences sufficient discomfort to interfere with or reduce his or her usual level of activity.

Severe: Significant impairment of functioning: the patient is unable to carry out usual activities.

Life-threatening: The patient's life is at risk from the adverse event. Adverse event data will be obtained from any information volunteered by the patient or through patient questioning. Additionally, adverse event data will also be collected through the use of a diary card.

Serious Adverse Events

A serious adverse event (SAE) is defined as any untoward medical occurrence that at any dose:
Results in death,
Is life-threatening (i.e., the patient was at risk of death at the time of the event.

This does not refer to an event that hypothetically might have caused death if it were more severe),
Requires inpatient hospitalisation or prolongation of existing hospitalisation,
Results in persistent or significant disability or incapacity,
Is a congenital anomaly or birth defect, or

Is another medically important condition (i.e., one that may not be immediately life threatening or result in death or hospitalisation, but is clearly of major clinical significance. It may jeopardise the patient, or may require intervention to prevent one of the other serious outcomes. Examples of such events are intensive treatment in an emergency room or at home for allergic bronchospasm, blood dyscrasias or convulsions that do not result in inpatient hospitalisations, or development of drug dependency or drug abuse.)

Clinical laboratory parameters

Routine haematology:
Haemoglobin
Haematocrit
Red cell count
White cell count
Neutrophils¹
Lymphocytes¹
Monocytes¹
Eosinophils¹
Basophils¹
Thrombocytes
Erythrocyte Sedimentation Rate¹
Electrophoresis¹
Urinalysis (dipstick):
Protein
Glucose
Ketones
PH
Blood
Clinical chemistry:
Sodium
Potassium
Urea
Creatinine
Total bilirubin
Total protein
Calcium
AST (SGOT) and/or ALT (SGPT)
Alkaline phosphatase
Glucose
Endocrinology:
Free Thyroxine (FT4)
Thyrotropin (thyroid stimulating hormone TSH)
Cortisol
Testosterone (men)
Follicle Stimulating Hormone (FSH)
Luteinising Hormone (LH)
Growth Hormone (GH)
Insulin-like growth factor I (IGF-1)
Insulin-like growth factor binding protein 3 (IGFBP -3)
Insulin
Growth Hormone antibodies
¹These tests will only be performed at the Baseline visit

All of the above-listed tests will be performed at the frequencies described in the study schedule below.

Blood Sample Collection and Serum Preparation

Local Laboratory Procedure: Haematology, Blood Chemistry, Endocrinology

30 ml of blood will be drawn (6 serum tubes of 4 ml each and 3 plasma EDTA tubes of 3 ml each). Samples for haemoglobin, leukocytes, thrombocytes, differentials and glucose will be analysed according to routine hospital procedure at the laboratory. All other samples will be stored at the Sahlgrenska hospital at −80° C. until assayed.

All the analyses will be performed at the laboratory of the Sahlgrenska University Hospital.

Serum Sample Collection for Anti-GH Antibodies

Using tubes without anticoagulant, two 5 ml blood samples are collected at the intervals indicated in the Study Plan (below). All serum samples will be labeled with the patient identification number and patient initials, collection date and time of blood sample. Samples may be collected when convenient (i.e. not at any specific times but within Study Day(s) indicated) but the exact time and date of sample collection must be recorded. The exact time of administration of the r-hGH dose preceding the sample collection must be recorded in the CRF. 8 ml of blood are withdrawn into a plain tube and serum is prepared by coagulation in the refrigerator (maximum time 30 minutes). Centrifuging is at room temperature 3500 RPM for 10 minutes (according to manufacturer specifications), serum collected and divided into 4 aliquots of 1 ml each. Samples are immediately frozen at or below −20° C. until shipment. One aliquot will be sent (frozen) to the central laboratory, three will be stored frozen at or below −20° C. at the study site as back-up samples. Samples will be shipped to the central laboratory by the Corporate CRA.

Study Schedule

| | Pre study | Study | | | | |
|---|---|---|---|---|---|---|
| Parameters | evaluation | Day 1 | Month 3 | Month 6 | Month 9 | Month 12 |
| Informed consent | X | | | | | |
| Medical review and history to | X | | | | | |
| satisfy eligibility criteria | | | | | | |
| Disease and therapy history | | X¹ | | | | |
| Demographic data | | X¹ | | | | |
| Physical exam and vital signs | | X¹ | X | X | X | X |
| Current disease related medical | | X¹ | | | | |
| condition | | | | | | |
| Haematology, chemistry and | | X¹ | X | X | X | X |
| urinalysis | | | | | | |
| Endocrinology | | X¹ | | X | | X |
| Antibodies to hGH | | X | | X | | X |
| Lumbar puncture for CSF | X | | | X | | X |
| analysis (GFAP, NFL) | | | | | | |
| NHP quality of life | | X | | X | | X |
| Functional assessments | | X | | X | | X |
| (UPDRS) | | | | | | |
| Measurement of PLM | | X | | | | X |
| Autonomic testing | | X | | X | | X |
| Study treatment | | X | X | X² | X | X |
| WHO toxicity grading | | | X | X | X | X |
| Adverse Event Reporting | | | X | X | X | X |
| Concomitant Medications and | | X | X | X | X | X |
| procedures | | | | | | |

¹If done within 14 days of Study Day 1, it is not necessary to repeat this procedure
²If the patient is significantly worse, the dose will be escalated to alternating daily injections of 1 mg and 0.5 mg

Unified Parkinson Disease Rating Scale (UPDRS)

The use of the UPDRS as a rating tool to follow the longitudinal course of Parkinson's Disease is a commonly accepted method. It is made up of the 1) Mentation, Behavior, and Mood, 2) ADL and 3) Motor sections. These are evaluated by interview. Some sections require multiple grades assigned to each extremity. A total of 199 points are possible. 199 represents the worst (total) disability, 0-no disability.

I. Mentation, Behavior, Mood

Intellectual Impairment
0-none
1-mild (consistent forgetfulness with partial recollection of events with no other difficulties)
2-moderate memory loss with disorientation and moderate difficulty handling complex problems
3-severe memory loss with disorientation to time and often place, severe impairment with problems
4-severe memory loss with orientation only to person, unable to make judgments or solve problems

Thought Disorder
0-none
1-vivid dreaming
2-“benign” hallucination with insight retained
3-occasional to frequent hallucination or delusions without insight, could interfere with daily activities
4-persistent hallucination, delusions, or florid psychosis.

Depression
0-not present
1-periods of sadness or guilt greater than normal, never sustained for more than a few days or a week
2-sustained depression for >1 week
3-vegetative symptoms (insomnia, anorexia, abulia, weight loss)
4-vegetative symptoms with suicidality

Motivation/lnitiative
0-normal
1-less of assertive, more passive
2-loss of initiative or disinterest in elective activities
3-loss of initiative or disinterest in day to say (routine) activities
4-withdrawn, complete loss of motivation
   II. Activities of Daily Living

Speech
0-normal
1-mildly affected, no difficulty being understood
2-moderately affected, may be asked to repeat
3-severely affected, frequently asked to repeat
4-unintelligible most of time

Salivation
0-normal
1-slight but noticeable increase, may have nighttime drooling
2-moderately excessive saliva, hay minimal drooling
3-marked drooling

Swallowing
0-normal
1-rare choking
2-occasional choking
3-requires soft food
4-requires NG tube or G-tube

Handwriting
0-normal
1-slightly small or slow
2-all words small but legible
3-severely affected, not all words legible
4-majority illegible

Cutting Food/Handing Utensils
0-normal
1-somewhat slow and clumsy but no help needed
2-can cut most foods, some help needed
3-food must be cut, but can feed self
4-needs to be fed

Dressing
0-normal
1-somewhat slow, no help needed
2-occasional help with buttons or arms in sleeves
3-considerable help required but can do something alone
4-helpless

Hygiene
0-normal
1-somewhat slow but no help needed
2-needs help with shower or bath or very slow in hygienic care
3-requires assistance for washing, brushing teeth, going to bathroom
4-helpless

Turning in Bed/Adjusting Bed Clothes
0-normal
1-somewhat slow no help needed
2-can turn alone or adjust sheets but with great difficulty
3-san initiate but not turn or adjust alone
4-helpless

Falling-Unrelated to Freezing
0-none
1-rare falls
2-occasional, less than one per day
3-average of once per day
4->1 per day

Freezing When Walking
0-normal
1-rare, may have start hesitation
2-occasional falls from freezing,
3-frequent freezing, occasional falls
4-frequent falls from freezin

Walking
0-normal
1-mild difficulty, day drag legs or decrease arm swing
2-moderate difficultly requires no assist
3-severe disturbance requires assistance
4-cannot walk at all even with assist

Tremor
0-absent
1-slight and infrequent, not bothersome to patient
2-moderate, bothersome to patient
3-severe, interfere with many activities
4-marked, interferes with many activities

Sensory Complaints Related to Parkinsonism
0-none
1-occasionally has numbness, tingling, and mild aching
2-frequent, but not distressing
3-frequent painful sensation
4-excruciating pain
   III. Motor Exam

Speech
0-normal
1-slight loss of expression, diction, volume
2-monotone, slurred but understandable, mod. impaired
3-marked impairment, difficult to understand
4-unintelligible

Facial Expression
0-Normal
1-slight hypomymia, could be poker face
2-slight but definite abnormal diminution in expression
3-mod. hypomimia, lips parted some of time
4-masked or fixed face, lips parted ¼ of inch or more with complete loss of expression

Tremor at Rest

Face
0-absent
1-slight and infrequent
2-mild and present most of time
3-moderate and present most of time
4-marked and present most of time

Right Upper Extremity (RUE)
0-absent
1-slight and infrequent
2-mild and present most of time
3-moderate and present most of time
4-marked and present most of time

LUE
0-absent
1-slight and infrequent
2-mild and present most of time
3-moderate and present most of time
4-marked and present most of time

RLE
0-absent
1-slight and infrequent
2-mild and present most of time
3-moderate and present most of time
4-marked and present most of time

LLE
0-absent
1-slight and infrequent
2-mild and present most of time
3-moderate and present most of time
4-marked and present most of time

Action or Postural Tremor

RUE
0-absent
1-slight, present with action
2-moderate, present with action
3-moderate present with action and posture holding
4-marked, interferes with feeding

LUE
0-absent
1-slight, present with action
2-moderate, present with action
3-moderate present with action and posture holding
4-marked, interferes with feeding

Rigidity

Neck
0-absent
1-slight or only with activation
2-mild/moderate
3-marked, full range of motion
4-severe

RUE
0-absent
1-slight or only with activation
2-mild/moderate
3-marked, full range of motion
4-severe

LUE
0-absent
1-slight or only with activation
2-mild/moderate
3-marked, full range of motion
4-severe

RLE
0-absent
1-slight or only with activation
2-mild/moderate
3-marked, full range of motion
4-severe

LLE
0-absent
1-slight or only with activation
2-mild/moderate
3-marked, full range of motion
4-severe

Finger Taps

Right
0-normal
1-mild slowing, and/or reduction in amp.
2-moderate impaired. Definite and early fatiguing, may have occasional arrests
3-severely impaired. Frequent hesitations and arrests.
4-can barely perform

Left
0-normal
1-mild slowing, and/or reduction in amp.
2-moderate impaired. Definite and early fatiguing, may have occasional arrests
3-severely impaired. Frequent hesitations and arrests.
4-can barely perform

Hand Movements (Open and Close Hands in Rapid Succession)

Right
0-normal
1-mild slowing, and/or reduction in amp.
2-moderate impaired. Definite and early fatiguing, may have occasional arrests
3-severely impaired. Frequent hesitations and arrests.
4-can barely perform

Left
0-normal
1-mild slowing, and/or reduction in amp.
2-moderate impaired. Definite and early fatiguing, may have occasional arrests
3-severely impaired. Frequent hesitations and arrests.
4-can barely perform

Rapid Alternating Movements (Pronate and Supinate Hands)

Right
0-normal
1-mild slowing, and/or reduction in amp.
2-moderate impaired. Definite and early fatiguing, may have occasional arrests
3-severely impaired. Frequent hesitations and arrests.
4-can barely perform

Left
0-normal
1-mild slowing, and/or reduction in amp.
2-moderate impaired. Definite and early fatiguing, may have occasional arrests
3-severely impaired. Frequent hesitations and arrests.
4-can barely perform

Leg Agility (tap heel on ground, amp should be 3 inches)

Right
0-normal
1-mild slowing, and/or reduction in amp.
2-moderate impaired. Definite and early fatiguing, may have occasional arrests
3-severely impaired. Frequent hesitations and arrests.
4-can barely perform

Left
0-normal
1-mild slowing, and/or reduction in amp.
2-moderate impaired. Definite and early fatiguing, may have occasional arrests
3-severely impaired. Frequent hesitations and arrests.
4-can barely perform

Arising From Chair (Pt. Arises with Arms Folded Across Chest)
0-normal
1-slow, may need more than one attempt
2-pushes self up from arms or seat
3-tends to fall back, may need multiple tries but can arise without assistance
4-unable to arise without help

Posture
0-normal erect
1-slightly stooped, could be normal for older person
2-definitely abnormal, mod. stooped, may lean to one side
3-severely stooped with kyphosis
4marked flexion with extreme abnormality of posture

Gait
0-normal
1-walks slowly, may shuffle with short steps, no festination or propulsion
2-walks with difficulty, little or no assistance, some festination, short steps or propulsion
3-severe disturbance, frequent assistance
4-cannotwalk

Postural Stability (Retropulsion Test)
0-normal
1-recovers unaided
2-would fall if not caught
3-falls spontaneously
4unable to stand

Body Bradykinesia/Hypokinesia
0-none
1-minimal slowness, could be normal, deliberate character
2-mild slowness and poverty of movement, definitely abnormal, or dec. amp. of movement
3-moderate slowness, poverty, or small amplitude
4-marked slowness, poverty, or amplitude
   Nottingham Health Profile

The use of this questionnaire is a commonly accepted method to measure quality of life.

Listed below are some problems people might have in their daily lives. Read the list carefully and put a tick in the box r under “YES” for any problem that applies to you at the moment. Tick the box under “NO” for any problem that does not apply to you. Please answer every question. If you are not sure whether to answer yes or no, tick which ever answer you think is most true at the moment.

| | YES | NO |
|---|---|---|
| I'm tired all the time | r | r |
| I have pain at night | r | r |
| Things are getting me down | r | r |
| I have unbearable pain | r | r |
| I take tablets to help me sleep | r | r |
| I've forgotten what it's like to enjoy myself | r | r |
| I'm feeling on edge | r | r |
| I find it painful to change position | r | r |
| I feel lonely | r | r |
| I can only walk about indoors | r | r |
| I find it hard to bend | r | r |
| Everything is an effort | r | r |
| I'm waking up in the early hours of the morning | r | r |
| I'm unable to walk at all | r | r |
| I'm finding it hard to make contact with people | r | r |
| The days seem to drag | r | r |
| I have trouble getting up and down stairs or steps | r | r |
| I find it hard to reach for things | r | r |
| I'm in pain when I walk | r | r |
| I lose my temper easily these days | r | r |
| I feel there is nobody I am close to | r | r |
| I lie awake for most of the night | r | r |
| I feel as if I'm losing control | r | r |
| I'm in pain when I'm standing | r | r |
| I find it hard to dress myself | r | r |
| I soon run out of energy | r | r |
| I find it hard to stand for long (eg at the kitchen | r | r |
| sink, waiting for a bus) | | |
| I'm in constant pain | r | r |
| It takes me a long time to get to sleep | r | r |
| I feel I am a burden to people | r | r |
| Worry is keeping me awake at night | r | r |
| I feel that life is not worth living | r | r |
| I sleep badly at night | r | r |
| I'm finding it hard to get on with people | r | r |
| I need help to walk about outside (eg a walking aid | r | r |
| or someone to support me) | | |
| I'm in pain when going up and down stairs or steps | r | r |
| I wake up feeling depressed | r | r |
| I'm in pain when I'm sitting | r | r |

Now please go back to the start and make sure that you have answered “YES” or “NO” to every question of the questionnaire.

Description of Posturo-Locomotor Manual Test

(Johnels B, Ingvarsson P. E, Thorselius M, Valls M, Steg G. Disability profiles and objective quantitative assessment in Parkinson's disease. Acta Neurol. Scand., 1989:79:227-238)

The Posturo-Locomotor Manual (PLM) test is a new method, designed to measure the degree of disability in Parkinson's disease. It utilizes an opto-electronic camera to record the body movements of freely moving patients. An automatic computer technique quantifies the motor performance to secure an objective evaluation.

Test Procedure

The PLM test requires that the subject picks up an object on the floor and walks forward to deliver it on a shelf placed at the height of his chin, thus forcing the body through postural changes and forward locomotion, and requiring a goal-directed arm-hand movement. By comparing with the performance of normal subjects of the same age, the test can be used to measure the degree of disturbance in postural, locomotive and manual motor acts, all elementary motor functions necessary for an independent life, and often defective in Parkinsonian patients.

The idea put forward is that patients with Parkinson's disease differ from each other in the degree and pattern of dopamine nerve cell degeneration in the brain, and that this in each case will cause varying degrees of postural, locomotor and manual disturbances, a disability profile and necessitate and individualized treatment. If so, the PLM-test should serve to record and quantify this profile for each patient as a base for a rational treatment

Description of Autonomic Testing

Holmberg B, Kallio M, Johnels B, Elam M. Cardiovascular Reflex Testing contributes to Clinical Evaluation and Differential Diagnosis of Parkinsonian Syndromes

All autonomic tests are performed at the department of clinical neurophysiology, where clinical routine tests of autonomic function are based on analysis of heart rate and BP changes in time domain. Subjects are placed and secured on a tilting table, and recordings of heart rate and BP are initiated. Both a continuous non-invasive finger BP measurement according to the volume-clamp principle (Finapres, Ohmeda Monitoring Systems, Englewood, Colo., USA), and an automatic sphygomanometric method (Sphygmomanometer BP -203Y, Nippon Colin Co, Muranaka, Komaki-City, Japan) with the cuff placed on the left upper arm, are used. Heart rate is calculated with a computer program, triggered by the pulse signal from the Finapres. Sinus arrhythmia is evaluated during 60 seconds periods, at rest and during controlled deep breathing (6 respiratory cycles per period), and calculated according to the formula: 100 (HRmax-HRmin)IHRmean

Orthostatic tests are performed subsequently, with the time used for raising the tilt table from horizontal position to 75° head-up tilt limited to two seconds. Quantitative HR and BP changes during eight minutes of tilt are based on sphygomanometrical data, since it has been noted that hydrostatic errors are difficult to avoid with the Finapres equipment, event if care is taken to place the finger cuff at heart level. In a few patients, a rapid and marked orthostatic blood pressure fall was qualitatively documented by the Finapressignal, whereas no reliable cuff pressure data could be acquired before the patients had to be returned to horizontal position due to pre-syncopal symptoms.

Diagnostic Criteria for MSA

Multiple System Atrophy

(Gilman S, Low P A, Quinn N et al. Consensus statement on the diagnosis of multiple system atrophy. J Auton Nerv Syst 1998;74:189-192)

TABLE 1
Clinical domains, features and criteria
used in the diagnosis of MSA

| | |
|---|---|
| I. | Autonomic and urinary dysfunction |
| A. | Autonomic and urinary features |
| 1. | Orthostatic hypotension by (20 mm Hg systolic or 10 mm |
| | Hg diastolic) |
| 2. | Urinary incontinence or incomplete bladder emptying |
| B. | Criterion for autonomic failure or urinary dysfunction in MSA |
| | Orthostatic fall in blood pressure by (30 mm Hg systolic or 15 mm |
| | Hg diastolic) or urinary incontinence (persistent, involuntary |
| | partial or total bladder emptying, accompanied by erectile |
| | dysfunction in men) or both |
| II. | Parkinsonism |
| A. | Parkinsonian features |
| 1. | Bradykinesia (slowness of voluntary movement with progressive |
| | reduction in speed and amplitude during repetitive actions) |
| 2. | Rigidity |
| 3. | Postural instability (not caused by primary visual, vestibular, |
| | cerebellar, or proprioceptive dysfunction) |
| 4. | Tremor (postural, resting or both) |
| B. | Criterion for parkinsonism in MSA |
| | Bradykinesia plus at least one of items 2 to 4 |
| III. | Cerebellar dysfunction |
| A. | Cerebellar features |
| 1. | Gait ataxia (wide based stance with steps of irregular length |
| | and direction) |
| 2. | Ataxic dysarthria |
| 3. | Limb ataxia |
| 4. | Sustained gaze-evoked nystagmus |
| B. | Criterion for cerebellar dysfunction in MSA |
| | Gait ataxia plus at least one of items 2 to 4 |
| IV. | Corticospinal tract dysfunction |
| A. | Corticospinal tract features |
| 1. | Extensor plantar responses with hyperreflexia |
| B. | Corticospinal tract dysfunction in MSA: no Corticospinal |
| | tract features are used in defining the diagnosis of MSA. |

A feature (A) is a characteristic of the disease and a criterion (B)
is a defining feature or composite of features required for diagnosis.

TABLE 2
Diagnostic categories of MSA

| | |
|---|---|
| I. | Possible MSA: One criterion plus two features from separate |
| | other domains. When the criterion is parkinsonism, a poor |
| | levodopa response qualifies as one feature (hence only one |
| | additional feature is required) |
| II. | Probably MSA: Criterion for: autonomic failure/urinary |
| | dysfunction plus poorly developed levodopa responsive |
| | parkinsonism or cerebellar dysfunction |
| III. | Definite MSA: Pathologically confirmed by the presence of a |
| | high density of glial cytoplasmic inclusions in association |
| | with a combination of degenerative changes in the nigrostriatal |
| | and olivopontocerebellar pathways. |

TABLE 3
Exclusion criteria for the diagnosis of MSA

| | |
|---|---|
| I. | History |
| | Symptomatic onset under 30 years of age |
| | Family history of a similar disorder |
| | Systemic diseases or other identifiable causes for features listed in |
| | Table 1 |
| | Hallucinations unrelated to medication |
| II. | Physical examination |
| | DSM criteria for dementia |
| | Prominent slowing of vertical saccades or vertical supranuclear |
| | gaze palsy |
| | Evidence of focal cortical dysfunction such as aphasia, alien limb |
| | syndrome, and parietal dysfunction |
| III. | Laboratory investigation |
| | Metabolic, molecular genetic and imaging evidence of an alternative |
| | cause of features listed in Table 1 |

Results After Six Months

Baseline Characteristics

18 patients out of a total of 43 randomised were included in this interim analysis for the purpose of safety (9 Placebo, 9 r-hGH). Of these 18 patients 12 were included in the efficacy analyses (6 Placebo, 6 r-hGH).

Demographics

No demographic differences were noted between the two groups. A slightly higher weight and BMI were found in the Placebo group but this should not be of any clinical relevance.

Disease Type

No difference in the distribution of disease type i.e. MSA-P or MSA-C was found between the treatment groups. However one thing which was noted was the overall distribution of the type of disease. In the ‘normal’ population of this disease area it would be expected to find approximately ⅔ patients with MSA-P and ⅓ MSA-C. However in the present analysis population an even split of 50% in each type was observed. Only one centre which followed the expected pattern. In the other two centers slightly more MSA-C than MSA-P was observed.

These differences were not of any clinical relevance given the good distribution between the treatment groups.

Disease Symptoms

The mean duration of time from MSA diagnosis to first dose of study drug was slightly lower in the r-hGH patients (approximately 1.5 yrs compared to 2 yrs in the placebo). However when the data were analyzed more closely, it was observed that in fact looking at the other symptoms used to diagnose MSA, the patients should have had the disease for about 3-5 years, not the 2 years stated.

Efficacy Parameters

For the purpose of efficacy evaluation only patients who completed their 6 month assessment were eligible i.e. those patient who dropped out prior to 6 months were excluded. As a result of this 6 patients were excluded, 3 in each treatment group. Two patients were reported to have died during their treatment phase, one from respiratory and circulatory failure and the other from an acute myocardial infarction. Three patients were withdrawn due to adverse events and two patients withdrew due to their own decision to leave the study.

Therefore for the purpose of efficacy evaluation 12 patients, 6 in each treatment group were included.

Motor Examination

On looking at the change in scores from baseline to 6 months there was an increase in the mean levels in both treatment groups. However it was seen that the level in the Placebo group had increased more than that in the r-hGH. The level of change seen in the r-hGH group was much better than would normally be expected.

Therefore it was concluded that there is a clinical difference emerging between the treatment groups in favour of r-hGH.

UPDRS Total Score

On looking at the change in scores from baseline to 6 months there was an increase in the mean levels in both treatment groups. However it was seen that the level in the Placebo group had increased more than that in the r-hGH. It was noted that this was probably a follow-on effect from the good result in the motor examination scores. An increase of approximately 10-15 points is normal and the mean change in the r-hGH group was slightly below this.

Therefore it was concluded that there is a clinical difference emerging between the treatment groups in favour of r-hGH.

Autonomic Tests

HRV at Deep Breathing

On looking at the change in the level of HRV at deep breathing from baseline to 6 months there was a decrease in the mean level of change in the Placebo group compared to an increase in the mean levels in the r-hGH group. In fact the mean level in the r-hGH group had nearly doubled from baseline to Month 6.

These results look very promising and indicate that there is a better regulation of heart rate with r-hGH in comparison to Placebo.

Therefore it was concluded that there is a clinical difference emerging between the treatment groups in favour of r-hGH.

Mean Arterial Pressure (MAP)

On looking at the change in the level of MAP at deep breathing from baseline to 6 months there was a decrease in the mean level of change in the Placebo group compared to an increase in the mean levels in the r-hGH group.

These results look very promising and indicate that there was a better regulation of pressure with r-hGH in comparison to Placebo.

Therefore it was concluded that there is a clinical difference emerging between the treatment groups in favour of r-hGH.

Nottingham Health Profile

Pain

On looking at the level of change in the pain score from the NHP from baseline to 6 months, there was a slight increase in the mean level of change in the Placebo group compared to very little change in the mean level of change in the r-hGH group. This could be indicative of a benefit of r-hGH.

Emotion

On looking at the level of change in the emotion score from the NHP from baseline to 6 months, there was very little change in the mean level of change in the Placebo group compared to a decrease in the mean level of change in the r-hGH group. This could be indicative of a benefit of r-hGH.

Physical

On looking at the level of change in the physical score from the NHP from baseline to 6 months, there was a slight increase in the mean level of change in the Placebo group compared to very little change in the mean level of change in the r-hGH group. This could be indicative of a benefit of r-hGH.

SBP Supine

On looking at the level of change in the supine blood pressure from baseline to 6 months an increase in the mean level was seen in both treatment groups. The level of increase in the Placebo group was slightly lower than that of the r-hGH group.

Therefore it was concluded that there is a clinical difference emerging between the treatment groups in favour of r-hGH.

SBP Standing

On looking at the level of change in the standing blood pressure from baseline to 6 months an increase in the mean level was seen in both treatment groups. The level of increase in the Placebo group was much lower than that of the r-hGH group. It was noted that this was in the same direction as the autonomics and again showed a possible benefit of r-hGH.

Therefore it was concluded that there is a clinical difference emerging between the treatment groups in favour of r-hGH.

### EFFICACY CONCLUSIONS

Overall there positive effect of r-hGH on the autonomic variables i.e. HRV, MAP and blood pressures, and an effect on the motor symptoms of the scales could be observed. Thus, hGH is proposed as a new treatment for multiple system atrophy.

### REFERENCES

1. Adelman et al, DNA 2:183 (1983)
2. Alba-Roth et al, J. Clin. Endocrin. Metab. 67:1186-1189 (1988)
3. Albertsson-Wikland et al, Acta Paediatr. Scand. 75:89 (1986)
4. Andersson K, Fuxe K, Eneroth P, Isaksoon O, Nyberg F, Roos P. Rat growth hormone and hypothalamic catecholamine nerve terminal systems. Evidence for rapid and discrete reductions in dopamine and noradrenaline levels in the median eminence of the hypophysectomized male rat. Eur J Pharmacol 1983;95:271-275
5. Altschul S F et al, J Mol Biol, 215, 403-410, 1990
6. Altschul S F et al, Nucleic Acids Res., 25:389-3402, 1997
7. Ausubel et al, Current Protocols in Molecular Biology, Greene Publications and Wiley lnterscience (New York, 1987-1998)
8. Bandmann O, Sweeney M G, Daniel S E, Wenning G K, Quinn N P, Marsden C D, Wood N W. Multiple system atrophy is genetically distinct from identified inherited causes of spinocerebellar degeneration. Neurology 1997;49:1598 -1604.
9. Becker et al, Biotechnol. Appl. Biochem. 10:326 (1988)
10. Bewly et al, Int. J. Peptide and Protein Res. 4:281-287 (1972)
11. Bowers et al, Endocrinology 128:2027-2035 (1991)
12. Burman P, Broman J E, Hetta J, Wiklund I, Erfurth E M, Hägg E, Karlsoon F A. Quality of life in adults with growth hormone (GH) deficiency: response to treatment with recombinant human GH in a placebo-controlled 21 month trial. J Clin Endocrinol Metab 1995;80:3585-90.
13. Camanni et al, Front. Neuroendocrinology 19:47-72 (1998)
14. Clemmons D R, Jones J I, Busby W H, Wright G: Role of insulin.like growth factor binding proteins in modifying IGF action Ann N Y Acad Sci. 1993 Aug 27;692:10-2lGilman S, Low P A, Quinn N et al. Consensus statement on the diagnosis of multiple system atrophy. J Auton Nerv Syst 1998;74:189-192
15. Chen et al, Genomics 4:479-497 (1989)
16. Daniel S E. The neuropathology and neurochemistry of multiple system atrophy. In: Mathias C J, Bannister R, eds. Autonomic failure: a textbook of the autonomic nervous system. 4th ed. Oxford: Oxford University Press; 1999. p. 321-328.
17. DeNoto et al, Nucleic Acids Res. 9:3719 (1981)
18. Devereux J et al, Nucleic Acids Res, 12, 387-395, 1984.
19. Elias Eriksson. Experimental psychoneuroendocrinology: Brain a adrenoceptor function and growth hormone release. Thesis, Goteborg University 1985
20. Felix et al, Int. J. Peptide Protein Res. 46:253-264 (1995)
21. Friend et al, Eur. J. Endocrinol. 137:377-386 (1997)
22. Gertler et al, Endocrinology 118:720 (1986)
23. Gilman S, Low P A, Quinn N et al. Consensus statement on the diagnosis of multiple system atrophy. J Auton Nerv Syst 1998;74:189-192
24. Ghigo et al, Ann. Med. 30:159-168 (1998)
25. Goeddel et al Nature, 281:544 (1979)
26. Graff et al, J. Biol. Chem. 257:2365 (1982)
27. Grantham, Science, Vol. 185, pp. 862-864 (1974).
28. Harvey S, Hull K L, Fraser R A. Mini-review; Growth Hormone: Neurocrine and neuroendocrine perspectives. Growth Reg 1993;3:161-171
29. Holmberg B, Rosengren L, Karlsson J E, Johnels B. Increased cerebrospinal fluid levels of neurofilament protein in progressive supranuclear palsy and multiple system atrophy compard with Parkinson's Disease. Mov Disorders 1998; 13:70-77
30. Hsiung et al, Biotechnology 7:267 (1989)
31. Hughes A J, Lees A J, Stern GM Apomorphine test to predict dopaminergic responsiveness in parkinsonian syndromes. Lancet 1990;336:32-34.
32. Hughes A J, Lees A J, Stern GM. Challenge test to predict the dopaminergic response in untreated Parkinson's disease. Neurology 1991;41:1723-1725.
33. Hughes A J, Colosimo C, Kleedorfer B, Daniel S E, Lees A J. The dopaminergic response in multiple system atrophy. J Neurol Neurosurg Psychiatry 1992;55:1009-1013c.
34. Johansson J -O, Wiren L, Bengtsson B -A. Growth Hormone and psychosocial and central nervous effects (in: Growth hormone in adults-physiological and clinical aspects, 2^{nd} edition, edited by Dr A Juul and Dr J O L Jorgensen). Cambridge University Press 2000:349-372
35. Jorgensson et al, Pharmacol. Toxicol. 63:129 (1988)
36. Kimber et al. Distinction of idiopathic Parkinsons's disease from multiple-system atrophy by stimulation of growth hormone release with clonidine. Lancet 1997;349:1877-1881.
37. Kimber J, Mathias C J, Lees A J, Bleasdale -Barr K, Chang H S, Churchyard A, Watson L. Physiological, pharmacological and neurohormonal assessment of autonomic function in progressive supranuclear palsy. Brain 2000; 123:1422-1430.
38. Klein C, Brown R, Wenning G, Quinn N. The “cold hands sign” in multiple system atrophy. Mov Disord 1997;12:514-518.
39. Kraft E, Schwartz J, Trenkwalder C, Vogl T, Pluger T, Oertel W. The combination of hypointense and hyperintense signal changes on T2-weighted magnetic resonance imaging sequences. A specific marker of multiple system atrophy? Arch Neurol 1999;56:225-228.
40. Lewis et al, Endocrinology 101:1587 (1977)
41. Lewis et al, J. Biol. Chem. 253:2679 (1978)
42. Lewis et al, Endocrinology 104:1256 (1979)
43. Lewis et al, Biochem. Biophys. Res. Comm. 92:511 (1980)
44. Lewis et al, J. Biol. Chem. 256:11645 (1981)
45. Litvan I, Agid Y, Calne D, Campbell G, Dubois B, Duvoisin R C, Goetz C G, Golbe L I, Grafman J, Growdon J H, Hallett M, Jankovic J, Quinn N P, Tolosa E, Zee DS. Neurology 1996 Jul;47(1):1 -9.
46. Litvan I, Goetz C G, Jankovic J, Wenning G K, Booth V, Bartko J J, McKee A, Jellinger K, Lai E C, Brandel J P, Verny M, Chaudhuri K R, Pearce R K, Agid Y. What is the accuracy of the clinical diagnosis of multiple system atrophy? A clinicopathologic study. Arch Neurol 1997 ;54:937-44.
47. Martial et al, Science 205:602-607 (1979)
48. Meinkoth J, Wahl G. Hybridization of nucleic acids immobilized on solid supports.Anal Biochem. 1984 May 1;138(2):267-84.
49. Moore et al, Endocrinology 122:2920 (1988)
50. Pearson W R, Methods in Enzymology, 183, 63-99, 1990
51. Pearson W R and Lipman D J, Proc Nat Acad Sci USA, 85, 2444-2448,1988
52. Polo K B, Jabbari B. Botulinum toxin-A improves the rigidity of progressive supranuclear palsy. Ann Neurol 1994;35:237-239.
53. Quinn N. Multiple System Atrophy-The nature of the beast. J Neurol, Neurosurg Psychiatry 1989; Special Supplement:78-89
54. Rosengren L E, Wikkelso C and Hagberg L. A sensitive ELISA for glial fibrillary acidic protein: Application in CSF of adults. J Neurosci Methods 1994;51:197-204.
55. Rosengren L E, Lycke J, Andersen O. Glial fibrillary acidic protein in CSF of multiple sclerosis patients: relation to neurological deficit. J Neurol Sci 1995; 133:61-65.
56. Rosengren L E, Karlsson J E, Karlsson J O, Persson L I, Wikkelso C. Patients with amyotrophic lateral sclerosis and other neurodegenerative diseases have increased levels of neurofilament protein in CSF. J Neurochem 1996;67:2013-2018.
57. Rosengren L E, Karlsson J E, Sjogren M, Blennow K, Wallin A. Neurofilament protein levels in CSF are increased in dementia. Neurology 1999;23;1090-1093.
58. Rossi P, Colosimo C, Moro E, Tonali P, Albanese A. Acute challenge with apomorphine and levodopa in parkinsonism. Eur Neurol 2000;43:95-101.
59. Schrag A, Kingsley D, Phatouris C, Mathias C J, Daniel S E, Quinn N P. Clinical usefulness of magnetic resonance imaging in multiple system atrophy. J Neurol Neurosurg Psychiatry 1998;65;65-71.
60. Singh et al, Endocrinology 94:883 (1974)
61. Smith et al, Science 260:1640-1643 (1998)
62. Smith and Waterman J Mol Biol, 147,195-197, 1981, Advances in Applied Mathematics, 2, 482-489,1981
63. Tannenbaum G S. Evidence for autoregulation of growth hormone secretion via the central nervous system. Endocrinology 1980; 107:2117-2120
64. Thorlacius-Ussing, Neuroendocrinology 43:233 (1987)
65. Thorner et al, Recent Prog. Horm. Res. 52:215-244 (1997)
66. Van der Ecken H, Adams R D, van Bogaert L. S triopallidal-nigral degeneration. A hitherto undescribed lesion in paralysis agitans. J Neuropathol Exp Neurol 1960; 19:159-61.
67. Wenning G K, Quinn N, Magalhaes C, Mathias C, Daniel S E. “Minimal change” Multiple System Atrophy. Mov Disord 1994; 9:161-166.
68. Wenning G K. Olivopontocerebellar atrophy. Mov Disord 1996;11:157-162.
69. Wenning G K, Tison F, Ben Shlomo Y, Daniel S E, Quinn N P. Multiple system atrophy: A Review of 203 Pathologically Proven Cases. Mov Disord 1997;12:133-147.

1. A method for the treatment and/or prevention of a Parkinsonism-Plus Syndrome comprising administering to a person in need thereof a substance selected from the group consisting of:
(a) human growth hormone;
(b) a variant of (a) which has at least 70% sequence identity thereto and which has agonistic activity on the hGH receptor;
(c) a variant of (a) having agonistic activity on the hGH receptor and which is encoded by a DNA sequence which hybridizes to the complement of the native DNA sequence encoding (a);
(d) a salt of (a), (b) or (c);
(e) human growth hormone releasing hormone (hGHRH);
(f) a variant of (e) which has at least 70% sequence identity thereto and which has agonistic activity on the hGHRH receptor;
(g) a variant of (e) having agonistic activity on the hGHRH receptor and which is encoded by a DNA sequence which hybridizes to the complement of the native DNA sequence encoding (e) under moderately stringent conditions;
(h) a salt of (e), (f) or (g);
(i) insulin-like growth factor (IGF);
(j) a nucleic acid encoding any one of (a)-(i); and
(k) combinations thereof.

2. The method of claim 1, wherein the Parkinsonism-Plus Syndrome is selected from the group consisting of Progressive Supranuclear Palsy (PSP), Multiple System Atrophy (MSA), Parkinson's-amyotrophic lateral sclerosis-dementia of Guam, Generalized Lewy body disease, Corticobasal ganglionic degeneration, Alzheimer's/Parkinson's overlap syndrome, Huntington's disease: rigid variant, Hallervorden-Spatz disease, and Gerstmann-Strausler syndrome.

3. (canceled)

4. (canceled)

5. The method of claim 1, wherein the substance is a naturally-occurring human growth hormone.

6. The method of claim 1, wherein the substance is recombinant human growth hormone.

7. (canceled)

8. The method of claim 1, wherein the variant comprises amino acids 177 to 191 of hGH.

9. The method of claim 1, wherein the variant is methionyl human growth hormone.

10. The method of claim 1, wherein the variant is lacking the 15 amino acid residues from Glu32 to Glu46 of hGH.

11. The method of claim 1, wherein the variant is lacking the first eight amino acid residues at the N-terminus.

12. The method of claim 1, wherein the variant is lacking the first 13 amino acid residues at the N-terminus.

13. The method of claim 1, wherein the substance comprises a dimer of human growth hormone selected from the group consisting of a disulfide dimer connected through interchain disulfide bonds, a covalent irreversible non-disulfide dimer, a non-covalent dimer, and mixtures thereof.

14. The method of claim 1, wherein the substance is chemically derivatized.

15. The method of claim 14, wherein the derivative is selected from the group consisting of:
(a) the substance is acetylated at the N-terminus;
(b) the substance is deaminated;
(c) the substance is sulfoxidized at one or more methionine residues; and
(d) the substance is derivatized at one or more amino acid side chains with a polyethylene glycol (PEG) moiety.

16. (canceled)

17. (canceled)

18. The method of claim 1, wherein the substance is administered at a dosage selected from the group consisting of:
(a) about 0.1 to 10 mg per person per day;
(b) about 0.5 to 6 mg per person per day;
(c) about 1 mg per person per day;
(d) a dosage administered daily;
(e) a dosage administered every other day;
(f) alternating daily dosages, wherein the first dosage is higher than the second dosage;
(g) alternating daily dosages, wherein the first dosage is about 1 mg per person and the second dosage is about 0.5 m per person;
(h) about 6 mg per person;
(i) about 5 mg per person; and
(j) about 4.5 mg per person.

19. (canceled)

20. (canceled)

21. (canceled)

22. (canceled)

23. (canceled)

24. (canceled)

25. The method of claim 14, wherein the substance is derivatized at one or more side chains of amino acid residues.

26. (canceled)

27. (canceled)

28. The method of 1, wherein the IGF is IGF-I or IGF-II.

29. The method of claim 1, wherein the substance is IGF and the patient is further administered IGFBP (Insulin-like Growth Factor Binding Protein) simultaneous, sequential, or separate from the IGF.

30. The method of claim 29, wherein the IGFBP is IGFBP3.

31. (canceled)

32. (canceled)

33. The method of claim 1, wherein the substance is administered in a manner selected from the group consisting of:
(a) the substance is administered subcutaneously;
(b) the substance is administered intramuscularly; and
(c) the substance is administered with an auto-injector.

34. (canceled)

35. (canceled)

36. The method of claim 1 wherein the nucleic acid is an expression vector.

37. A method for the treatment and/or prevention of a Parkinsonism-Plus Syndrome, comprising administering to a person in need thereof a cell, wherein the cell produces a substance capable of treating or preventing a Parkinsonism-Plus Syndrome according to the method of claim 1.

38. (canceled)
